SEQUENCE LISTING (13 sequences)

SEQ ID NO: 1 - DNA, 1284 bp, Homo sapiens
atgggcggggatctggtgcttggcctgggggccttgagacgccgaaagcgcttgctggagcaggagaagtctctggccggctgggcactggtgctggcaggaactggcattggactcatggtgctgcatgcagagatgctgtggttcggggggtgctcgtgggcgctctacctgttcctggttaaatgcacgatcagcatttccaccttcttactcctctgcctcatcgtggcctttcatgccaaagaggtccagctgttcatgaccgacaacgggctgcgggactggcgcgtggcgctgaccgggcggcaggcggcgcagatcgtgctggagctggtggtgtgtgggctgcacccggcgcccgtgcggggcccgccgtgcgtgcaggatttaggggcgccgctgacctccccgcagccctggccgggattcctgggccaaggggaagcgctgctgtccctggccatgctgctgcgtctctacctggtgccccgcgccgtgctcctgcgcagcggcgtcctgctcaacgcttcctaccgcagcatcggcgctctcaatcaagtccgcttccgccactggttcgtggccaagctttacatgaacacgcaccctggccgcctgctgctcggcctcacgcttggcctctggctgaccaccgcctgggtgctgtccgtggccgagaggcaggctgttaatgccactgggcacctttcagacacactttggctgatccccatcacattcctgaccatcggctatggtgacgtggtgccgggcaccatgtggggcaagatcgtctgcctgtgcactggagtcatgggtgtctgctgcacagccctgctggtggccgtggtggcccggaagctggagtttaacaaggcagagaagcacgtgcacaacttcatgatggatatccagtataccaaagagatgaaggagtccgctgcccgagtgctacaagaagcctggatgttctacaaacatactcgcaggaaggagtctcatgctgcccgcaggcatcagcgcaagctgctggccgccatcaacgcgttccgccaggtgcggctgaaacaccggaagctccgggaacaagtgaactccatggtggacatctccaagatgcacatgatcctgtatgacctgcagcagaatctgagcagctcacaccgggccctggagaaacagattgacacgctggcggggaagctggatgccctgactgagctgcttagcactgccctggggccgaggcagcttccagaacccagccagcagtccaagtag

SEQ ID NO: 2 - DNA, 1469 bp, Mus musculus
ctgggcaggaagctggctgagccccaagacctcaggggccatgggcggggagctggtgactggcctgggggccctgagacggagaaagcgcctgctggagcaggagaagagggtggccggctgggcgttggtgctggcgggaactggcatcggactcatggttctgcacgctgagatgttgtggttcctgggctgcaagtgggtgctgtacctgctcctggttaagtgtttgatcaccctgtccactgccttcctcctttgtcttattgtggtcttccatgccaaggaggtccagctgttcatgactgacaacgggctccgggactggcgcgtggcgctgacccggcggcaggtggcgcagatcctgctggagctgttggtgtgcggggtgcacccggtgcccctacggagcccgcactgcgccctggcgggggaggccaccgacgcgcagccctggccgggtttcctgggcgaaggcgaggcgttgctgtccctggccatgctcctgcgtctctacctggtgccccgcgcggtgctgctgcgcagcggggtcctgctcaacgcgtcctaccgcagcatcggggcgctcaaccaagtccgcttccgccactggttcgtggccaagctgtacatgaacacgcacccgggtcgcctgctgctgggcctcacgctgggtctctggctcaccacagcttgggtgctgtctgtggctgagaggcaggctgtcaatgccacggggcacctcacagacacactgtggctgattccgatcacattcctgaccattggctatggggacgtggtacctggcaccatgtggggcaagattgtctgcctgtgcaccggagtcatgggggtctgctgcacagctctcctggtggctgtggtggctcggaagctggagttcaacaaggcggagaaacacgtgcacaacttcatgatggacatccattatgccaaagagatgaaggagtcagcggcgcggctgctgcaggaagcctggatgtactacaagcacactcgaaggaaggactcccgggctgcccgcagacatcagcgcaagatgctggccgccatccacacgttccgccaggtacggctgaaacaccggaagctccgggaacaagtgaattccatggtggacatctccaagatgcacatgatcctgtgcgacctgcagctgggtctcagctcctcgcaccgtgccctggagaagagaatcgacggtctggcaggaaagctggatgccctgacagagctgctcggcactgctctgcagcaacagcagctaccagaacccagtcaggaggccacatagctccacatgaactcacagaagaaccaggctaagtacccaaggaccgagctcaaggacatgctccctgccaattccgaccaagccccacgataaatcacctcaagatgccaggacccaagtggatccacgttgaggtgcatggactctggtg

SEQ ID NO: 3 - protein, 427 aa, Homo sapiens
MGGDLVLGLGALRRRKRLLEQEKSLAGWALVLAGTGIGLMVLHAEMLWFGGCSWALYLFLVKCTISISTFLLLCLIVAFHAKEVQLFMTDNGLRDWRVALTGRQAAQIVLELVVCGLHPAPVRGPPCVQDLGAPLTSPQPWPGFLGQGEALLSLAMLLRLYLVPRAVLLRSGVLLNASYRSIGALNQVRFRHWFVAKLYMNTHPGRLLLGLTLGLWLTTAWVLSVAERQAVNATGHLSDTLWLIPITFLTIGYGDVVPGTMWGKIVCLCTGVMGVCCTALLVAVVARKLEFNKAEKHVHNFMMDIQYTKEMKESAARVLQEAWMFYKHTRRKESHAARRHQRKLLAAINAFRQVRLKHRKLREQVNSMVDISKMHMILYDLQQNLSSSHRALEKQIDTLAGKLDALTELLSTALGPRQLPEPSQQSK

SEQ ID NO: 4 - protein, 425 aa, Mus musculus
MGGELVTGLGALRRRKRLLEQEKRVAGWALVLAGTGIGLMVLHAEMLWFLGCKWVLYLLLVKCLITLSTAFLLCLIVVFHAKEVQLFMTDNGLRDWRVALTRRQVAQILLELLVCGVHPVPLRSPHCALAGEATDAQPWPGFLGEGEALLSLAMLLRLYLVPRAVLLRSGVLLNASYRSIGALNQVRFRHWFVAKLYMNTHPGRLLLGLTLGLWLTTAWVLSVAERQAVNATGHLTDTLWLIPITFLTIGYGDVVPGTMWGKIVCLCTGVMGVCCTALLVAVVARKLEFNKAEKHVHNFMMDIHYAKEMKESAARLLQEAWMYYKHTRRKDSRAARRHQRKMLAAIHTFRQVRLKHRKLREQVNSMVDISKMHMILCDLQLGLSSSHRALEKRIDGLAGKLDALTELLGTALQQQQLPEPSQEAT

SEQ ID NO: 5 - DNA, 265 bp, Homo sapiens
cattcctgaccatcggctatggtgacgtggtgccgggcaccatgtggggcaagatcgtctgcctgtgcactggagtcatgggtgtctgctgcacagccctgctggtggccgtggtggcccggaagctggagtttaacaaggcagagaagcacgtgcacaacttcatgatggatatccagtataccaaagagatgaaggagtccgctgcccgagtgctacaagaagcctggatgttctacaaacatactcgcaggaaggagtctca

SEQ ID NO: 6 - DNA, 16 bp, Mus musculus
ctggcgggaactggca

SEQ ID NO: 7 - DNA, 17 bp, Mus musculus
cacccacttgcagccca

SEQ ID NO: 8 - DNA, 19 bp, Homo sapiens
gcgctctcaatcaagtccg

SEQ ID NO: 9 - DNA, 22 bp, Homo sapiens
gtgttcatgtaaagcttggcca

SEQ ID NO: 10 - DNA, 22 bp, Homo sapiens
catgggtgtgaaccatgagaag

SEQ ID NO: 11 - DNA, 21 bp, Homo sapiens
ctaagcagttggtggtgcagg

SEQ ID NO: 12 - DNA, 19 bp, Mus musculus
atcaacgggaagcccatca

SEQ ID NO: 13 - DNA, 20 bp, Mus musculus
gacatactcagcaccggcct

(19) United States
(12) Patent Application Publication
(10) Pub. No.: US 2004/0248099 A1
(43) Pub. Date: Dec. 9, 2004

(54) Use of intermediate -conductance potassium channels and modulators for diagnosing and treating diseases having disturbed keratinocyte activity

(76) Inventors: Andreas Goppelt, Munchen (DE); Christian Alzheimer, Kiel (DE); Heidi Kogel, Munchen (DE)

Correspondence Address: CLARK & ELBING LLP, 101 FEDERAL STREET, BOSTON, MA, 02110, US

(21) Appl. No.: 10/451,805

(22) PCT Filed: Dec. 27, 2001
(86) PCT No.: PCT/EP01/15317

(30) Foreign Application Priority Data
Dec. 28, 2000 (DE) 100 65 475.4

(51) Int. Cl. C12Q 1/68; G01N 33/53; G01N 33/567

(52) U.S. Cl. 435/6; 435/7.2

(57) ABSTRACT

The invention relates to the use of intermediate-conductance, calcium-activated potassium channels and/or the nucleic acids coding for the same, from humans or mice, for the diagnosis, prevention and/or treatment of illnesses associated with disturbed keratinocyte activity. The invention also relates to the use of the same for identifying pharmacologically active substances. The invention further relates to the use of modulators of intermediate-conductance, calcium-activated potassium channels for the diagnosis, prevention and/or treatment of illnesses associated with disturbed keratinocyte activity.

The invention relates to the use of human or mouse intermediate-conductance calcium-activated potassium channels and/or their encoding nucleic acids for diagnosing, preventing and/or treating diseases which are connected with disturbed keratinocyte activity, and to their use for identifying pharmacologically active substances. In addition, the invention relates to the use of modulators of intermediate-conductance calcium-activated potassium channels for diagnosing, preventing and/or treating diseases which are connected with disturbed keratinocyte activity.

Many diseases of the skin are connected with disturbed keratinocyte activity. The importance of the keratinocytes for maintaining normal physiological processes in the skin during wound healing can be retraced by way of example: the wound healing process is an extremely complex process which is characterized by the phases of blood coagulation in the region of the wound, recruitment of inflammatory cells, reepithelialization and tissue reorganization. Blood coagulation results in the formation of a fibrin matrix which serves to fill the cavity arising from the wound and provides a matrix through which the cells can immigrate. Chemoattractants which have become deposited in the matrix then recruit neutrophils and monocytes which initiate an inflammatory reaction. There then comes the central step of reepithelialization for the purpose of once again forming a protective epithelial barrier over the wound. This is accomplished by keratinocytes, which constitute the majority of the epidermal cells and are only found in the skin. In order to achieve reepithelialization, the keratinocytes change [lacuna] integrin composition at the cell surface, as a result of which they detach themselves from their initial location and are able to migrate over the wound. There they reattach themselves, proliferate and finally differentiate. Keratinocytes secrete matrix metalloproteinases and collagenases which break down proteins in the surrounding tissue and thereby facilitate migration in the wound tissue. In addition, keratinocytes secrete growth factors such as VEGF, KAF, TGF-beta and TGF-alpha. These growth factors in turn stimulate cell proliferation and the formation of components of the extracellular matrix and mediate the growth of new blood vessels into the wound bed. During the subsequent tissue reorganization, which can last for up to 2 years after injury, there is a continuous process of collagen synthesis, of renewed breakdown of collagen, of squamous differentiation of the keratinocytes and the formation of a scar.

This overview of the wound healing process makes clear that wound healing which proceeds normally requires complex spatial and chronological changes in keratinocyte activity, such as migration, proliferation and differentiation. Disturbances in keratinocyte activies can therefore lead to disturbances in wound healing and to other skin diseases. Examples of diseases with which dysfunctional keratinocytes have been connected are psoriasis, contact dermatitis, atopic eczema, ulcera, vitiligo, hyperkeratoses, actinic keratoses and acne.

Disturbances in the wound healing or in the scar, such as the formation of hypertrophic scars or keloids, can also occur after the wound has closed. A connection with keratinocyte activity has also been suggested in the case of these diseases. Thus, keratinocytes derived from hypertrophic scars which were formed following burn wounds were demonstrated to exhibit a higher degree of proliferation, differentiation and activation than did keratinocytes derived from intact skin.

Skin diseases, disturbed wound healing and cosmetic impairment are problems which arise during aging. Thus, what is termed geriatric skin is characterized by thinning of the epidermis and consequently of the protective barrier. In addition, the number of enzymically active melanocytes declines by 10-20% every 10 years in humans from the age of 25-30 onwards, resulting in exposure to radiation increasing. In connection with this, the pigmentation of old skin is frequently heterogeneous; both local loss of pigmentation and hyperpigmentation are observed. This has been attributed, inter alia, to modification of the melanocyte/keratinocyte interaction. Examples of frequently occurring pigment lesions are lentigo and ephelides.

Only very little is known about the molecular principles which underlie skin diseases and wound healing and consequently also disturbances in wound healing. This is due to the enormous complexity of the skin and of the diseases which are associated with it.

Thus, therapies which have to date been developed for intervening in wound healing disturbances are not particularly satisfactory. Established forms of therapy are limited to physically supporting the wound healing (e.g. bandages, compresses or gels) or transplanting skin tissues, cultured skin cells and/or matrix proteins. In recent years, growth factors have been tested for their ability to ameliorate wound healing without, however, significantly improving on conventional therapy. The diagnosis of wound healing disturbances is also based on optical analyses of the skin which are not particularly informative since a deeper understanding of gene regulation during wound healing is thus far lacking.

Potassium channels constitute a heterogeneous group of proteins which play an important role in regulating the membrane potential and consequently also the secondarily energetized transmembrane transport of molecules. In addition, they frequently play a role in cell proliferation (Nilius and Droogmans; 1994, News in Physiol. Sci., 9: 105-110). In some cases, potassium channels have been connected with genetically transmissible diseases (Curran, 1998, Curr. Opin. in Biotech., 9: 565-572). This fact, and also the advantages of the potassium channels that they can be used for developing robust assay systems for in-vitro characterization and for high throughput screening, explain the attractiveness of potassium channels as a target for developing drugs (Curran, see above).

The potassium channel family is a very heterogeneous group which can be divided into five subfamilies: the voltage-activated potassium channels (Kv), the long QT related potassium channels, the inward rectifying potassium channels and the calcium-activated potassium channels. While a large number of publications mention the voltage-dependent potassium channels as being suitable targets for treating skin diseases (WO 99/07411; WO 97/18332; WO 99/25703), no drugs for treating diseases which are connected with disturbed keratinocyte activity have so far been developed.

The calcium-activated potassium channels are divided into three well characterized subgroups: the small-conductance (SK); the intermediate-conductance (IK) and the big-conductance (BK) potassium channels. These subgroups differ in their voltage sensitivity and calcium sensitivity, their expression profile, their biological function and their pharmacological properties.

Homologs of the IK channel have been identified both in humans (hIK1 or hsk4; Ishii et al. 1997 Proc. Natl. Acad. Sci. USA 94: 11651-11656; Joiner et al., 1997, Proc. Natl. Acad. Sci. USA, 94: 11013-11018; WO 99/03882; WO 98/11139) and in mice (mIK1; Vandorpe et al., 1998, J. Biol. Chem. 273: 21542-21553). Because they have the same properties, it is assumed that the Gardos channel, which has been characterized only electrophysiologically and pharmacologically in erythrocytes, is identical with hIK1 (Ishii et al. 1997 Proc. Natl. Acad. Sci. USA 94: 11651-11656). The Gardos channel plays an important function in the strength with which the sickle cell anemia phenotype is expressed by exerting an influence on the dehydration and volume of diseased erythrocytes (Curran, see above). In the mouse, the cDNA encoding the IK channel has been isolated from erythroleukemia cells (Vandorpe et al., see above). This study showed that the expression of mIK1 is increased during the differentiation of ES cells into erythroid cells. In addition, it was possible to inhibit the proliferation and differentiation with inhibitors of the ion channel.

It is striking that, in humans, the IK channel, in contrast to SK channels, is only expressed in a few cell types and organs (WO 99/03882; Ishii et al. 1997 Proc. Natl. Acad. Sci. USA 94: 11651-11656; Joiner et al., 1997, Proc. Natl. Acad. Sci. USA, 94: 11013-11018): all the publications show that expression is restricted to organs which cannot be excited electrically, such as placenta and prostate, whereas no expression is detected in the brain or in the heart. This is reflected in the school of thought that expression of hIK in humans is restricted to cells of the blood (e.g. B lymphocytes, T lymphocytes and erythrocytes) and of the vascular system (e.g. endothelial cells of the kidney and the mesentery and capillary endothelial cells in the brain (Kohler et al., 2000, Circ. Res. 87: 496-503) (WO 00/34248). The IK1 channel has also been shown to be expressed in cells of the urinary bladder and the colon (Ohya et al., 2000, Jpn. J. Pharmacol. 84: 97-100; Warth et al., 1999, Pflugers Arch. 438: 437-444). No expression of hIK1 or mIK1 has thus far been detected in skin or keratinocytes.

In addition to employing it for modulating sickle cell anemia, it has been proposed that hIK1 be used for diagnosing, preventing or treating diseases which are connected with dysfunctional leukocytes (WO 99/25347). In addition, inhibitors of the IK channel are used in connection with developing drugs for treating diarrhea and cystic fibrosis. In recent years, a large number of specific modulators of IK activity, which can be used pharmaceutically, have been developed (Syme et al., 2000, Am. J. Physiol. 278: C570-581; WO 99/25347; WO 00/33834; WO 00/34228; WO 00/34248; WO 00/37422).

The object of the invention was to make available polypeptides, and/or their encoding nucleic acids, which are involved in diseases which are connected with disturbed keratinocyte activity and whose use decisively improves diagnosis, prevention or treatment and the identification and development of pharmaceuticals which are effective in diseases which are connected with disturbed keratinocyte activity.

An additional object of the invention was to provide modulators of the activity of the polypeptides according to the invention, the use of which modulators decisively improves diagnosis, prevention or treatment and the identification and development of pharmaceuticals which are effective in diseases which are connected with disturbed keratinocyte activity.

When keratinocytes were stimulated with ATP, bradykinin and histamine, which are released in the skin in association with diseases which are connected with disturbed keratinocyte activity, the keratinocytes were found to be hyperpolarized for a long period. Surprisingly, it was possible to identify, as the mediator of this electrophysiological response, an intermediate-conductance calcium-activated potassium channel which had previously not been detected in the skin or in keratinocytes and which was for the first time brought into connection with disturbed keratinocyte activity. In addition, it was possible, for the first time, to establish a connection between the level of expression and/or activity of an intermediate-conductance calcium-activated potassium channel and potassium channel and the diagnosis, prevention and/or treatment of diseases which are connected with disturbed keratinocyte activity, in particular wound healing disturbances and/or psoriasis. The polypeptides, and their encoding nucleic acids, do not belong to the previously known targets for diagnosing, for example the indication and/or treatment, such as the modulation, and/or preventing diseases which are connected with disturbed keratinocyte activity, or for identifying pharmacologically active substances, which means that completely novel therapeutic approaches result from this invention. In addition, modulators of the polypeptides according to the invention, such as inhibitors or activators, have not been used in diagnosing, preventing and/or treating diseases which are connected with disturbed keratinocyte activity, which means that completely novel therapeutic approaches result.

The object is therefore achieved by using the human (hIK1 or hSK4; SEQ ID No. 3; Ishii et al. 1997 Proc. Natl. Acad. Sci. USA 94: 11651-11656; Joiner et al., 1997, Proc. Natl. Acad. Sci. USA, 94: 11013-11018; WO 99/03882; WO 98/11139) or mouse (mIK1; SEQ ID No. 4; Vandorpe et al. 1998, J. Biol. Chem. 273: 21542-21553) intermediate-conductance calcium-activated potassium channel IK1 polypeptide, or functional variants thereof, and/or nucleic acids encoding them, or variants thereof, for diagnosing, preventing and/or treating diseases which are connected with disturbed keratinocyte activity or for identifying pharmacologically active substances.

In addition, the object is achieved by using modulators of IK1 activity for diagnosing, preventing and/or treating diseases which are connected with disturbed keratinocyte activity.

None of the ion channels or their encoding nucleic acids has previously been reported to be connected with diseases which are connected with disturbed keratinocyte activity, for example in association with disturbed wound healing, nor has such a connection been suggested. It was therefore unexpected that these compounds can be used in accordance with the invention. The accession numbers of the polypeptides employed in accordance with the invention, and of their cDNAs, are listed in Table 1.

Furthermore, none of the modulators of the polypeptides according to the invention has previously been reported to be connected with skin diseases, for example in association with disturbed wound healing and/or psoriasis, nor has such a connection been suggested. It was therefore unexpected that these compounds can be used in accordance with the invention.

In addition to this, it has not been disclosed that the modulators of the activity of the polypeptides which can be used in accordance with the invention can be employed for diagnosing, preventing and/or treating diseases which are connected with disturbed keratinocyte activity, in particular wound healing disturbances and/or psoriasis, which means that this invention results in completely novel therapeutic approaches.

The polypeptides which are used in accordance with the invention can furthermore be characterized by the fact that they are prepared synthetically. Thus, the entire polypeptide, or parts thereof, can be synthesized, for example using classical synthesis (Merrifield technique). Parts of the above-described polypeptides are suitable, in particular, for obtaining antisera which can be used to screen suitable gene expression libraries in order, in this way, to obtain further functional variants of a polypeptide which can be used in accordance with the invention.

‘Keratinocyte activity’ is understood as meaning the state of a keratinocyte which is defined by the parameters proliferation rate and the degree of differentiation of the keratinocyte under investigation. The skilled person is familiar with methods for determining the parameters (de Fries and Mitsuhashi, 1995, J. Clin. Lab. Anal. 9:89-95; Perros and Weightman, 1991, Cell Prolif. 24:517-23; Savino and Dardenne, 1985, J. Immunol. Methods 85:221-6; Schulz et al., 1994, J. Immunol. Methods 167:1-13; Frahm et al., 1998, J. Immunol. Methods 211: 43-50; Rosenthal et al., 1992, J, Invest, Dermatol, 98:343-50). The degree of differentiation can, for example, be determined using suitable stains with which the skilled person is familiar.

‘Disturbed’ keratinocyte activity is understood as meaning a keratinocyte activity of a keratinocyte in the skin which differs from the keratinocyte activity of keratinocytes in normal, intact skin. For example, the activity of the ‘disturbed’ keratinocytes can differ from that of normal keratinocytes as a result of an increased or reduced proliferation rate or as the result of premature, accelerated, decelerated or delayed differentiation or differentiation which is not present. Examples of diseases which are connected with disturbed keratinocyte activity are contact dermatitis, atopic eczema, vitiligo, hyperkeratoses, actinic keratoses, hypertrophic scars, keloids, lentigo, ephelides and geriatric skin. In addition, wounds, for example normally healing wounds and poorly healing wounds, in particular ulcera, are, in particular, preferred diseases within the meaning of the present invention which are characterized by disturbed keratinocyte activity. A particularly preferred disease is psoriasis.

The term ‘regulation’ is understood, for example, as meaning an increase or decrease in the quantity and/or activity of the polypeptides or nucleic acids which encode them, with it being possible for this change to take place, for example, at the transcriptional, translational and posttranscriptional or posttranslational level.

The term ‘functional variants’ is to be understood as meaning polypeptides which, for example like the polypeptides which can be used in accordance with the invention, are connected with disturbed keratinocyte activity and/or exhibit structural features of the polypeptides which can be used in accordance with the invention.

Functional variants also include fusion proteins of the IK channels having a moiety of approx. 1-300, preferably approx. 1-200, particularly preferably approx. 1-150, in particular approx. 1-100, especially approx. 1-50, foreign amino acids, which can be linked to the polypeptide chain of the IK channels N-terminally, C-terminally or as an insertion. Furthermore, an IK channel which can be used in accordance with the invention can be linked to a green fluorescent protein or variants thereof.

Functional variants of the polypeptides can also be parts of the polypeptide which is used in accordance with the invention, having a length of at least 6 amino acids, preferably having a length of at least 8 amino acids, in particular having a length of at least 12 amino acids, very particularly having a length of at least 20 amino acids, especially having a length of 40 amino acids, most preferably having a length of at least 80 amino acids. Also included are deletions of the polypeptide in the range of approx. 1-60, preferably of approx. 1-30, in particular of approx. 1-15, especially of approx. 1-5, amino acids. For example, the first amino acid methionine can be missing without the function of the polypeptide being significantly altered.

Within the meaning of the present invention, ‘functional variants’ are polypeptides which exhibit a sequence homology, in particular a sequence identity, of approx. 50%, preferably approx. 60%, in particular approx. 70%, particularly preferably approx. 90%, most preferably 95%, with one of the IK channels having the amino acid sequence as depicted in either SEQ ID No. 3 or SEQ ID No. 4. Accordingly, the corresponding polypeptides which are derived from other organisms than humans or the mouse, preferably from nonhuman mammals, such as monkeys, pigs and rats, are also examples of these functional variants. Other examples are polypeptides which are encoded by different alleles of the gene, in different individuals or in different organs of an organism.

‘Sequence identity’ is understood as meaning the congruence (=% positive) which exists between two sequences and which is determined using BlastP 2.0.1, for example, in the case of polypeptide sequences and using BlastN 2.0.14, for example, in the case of polynucleotide sequences, with the filter being set at ‘off’ (Altschul et al., 1997, Nucleic Acid Res. 25: 3389-3402). For example, BlastP 2.0.1 is used for determining the similarity between two polypeptides, with the filter being set at ‘off’ and BLOSUM being 62.

Functional variants also include, for example, polypeptides which are encoded by nucleic acids which are isolated from non-skin-specific tissue, e.g. embryonic tissue, but which, following expression in a keratinocyte, possess the described functions.

In order to establish whether a polypeptide is a functional variant of a polypeptide which can be used in accordance with the invention, it is possible to employ functional tests to compare the activity of the polypeptide to be examined with the activity of a polypeptide, as depicted in SEQ ID No. 3 or SEQ ID No. 4, which can be used in accordance with the invention. On the assumption that the polypeptide to be examined meets the requirements a a functional variant at the level of sequence identity, the polypeptide to be examined is then a functional variant when its activity in the functional test turns out to be similar or identical to that of the polypeptide which can be used in accordance with the invention.

These functional tests include, for example, the application of an expression vector which contains a nucleic acid encoding the polypeptide to be examined, or the application of the actual polypeptide to be examined, or of an antibody which is directed against the polypeptide to be examined, or of an antisense polynucleotide, in wounds. Following incubation, for example of an expression vector, the progress of the wound healing is compared when administering the different expression vectors, containing either a nucleic acid encoding the polypeptide to be examined or a nucleic acid encoding a polypeptide which can be used in accordance with the invention, or an expression vector without insert. These functional tests can also be applied, for example, to the activity of the polypeptide to be examined in connection with disturbances in wound healing, for example in connection with poorly healing, dexamethasone-treated or diabetic wounds in animals. Thus, the application of PDGF-A and PDGF-B polypeptides to poorly healing wounds in rabbits led, for example, to a comparable improvement in the healing of the wounds (J. Surg. Res., 2000, 93: 230-236). Comparable tests can be carried out in the case of skin diseases, for example psoriasis. In this case, an expression vector which contains a nucleic acid encoding the polypeptide to be examined, or the actual polypeptide to be examined, or an antibody directed against the polypeptide to be examined, or an antisense oligonucleotide, is applied, for example, to an affected portion of human skin, which has been transplanted to SCID mice, and the course of the skin disease, for example the healing, is determined and compared with the course of the skin disease in a control experiment. In the case of psoriasis, this can also include, for example, antiproliferation studies in what are termed mouse tail tests. In this case, use is made of the fact that the normal histological structure of the skin of a mouse tail strongly resembles that of human skin which is affected with psoriasis: thus, tail skin possesses what are termed hinge regions, where hair strands come out from under the scales; at these sites, the skin is orthokeratotic and possesses a granular layer like normal skin. After a polypeptide to be examined has been added, histological sections of the tail skin are measured for the formation of a granulation layer, the induction of orthokeratotic tissue in parakeratotic regions and the thickness of the epidermis (Jarret et al., 1970, Br J Dermatol 82: 187-199). This method can also be quantified by measuring sagital sections after staining with hematoxylin/eosin (Bosman et al., 1992, Skin Pharmacol 5: 41-48; Bosman et al., 1994, Skin Pharmakol 7: 324-334). In addition, the antiinflammatory effect of the polypeptide to be examined can be employed by means of topical administration to mice in which edemas have been induced with oxazolone or to pigs in which allergic skin reactions have been induced with DNFB (Meingassner et al., 1997, Br J Dermatol 137: 568-576). Functional tests also involve measuring the effect of the polypeptide to be examined on the extent of the skin scaling. Thus, measurements of transepidermal water loss (TEWL) in psoriatric patients verify that the pathologically altered epithelial layers have lost their barrier function for water (Frodin et al., 1988, Acta Derm Venerol 68: 461-467, see example).

The term ‘encoding nucleic acid’ refers to a DNA sequence which encodes an isolatable, bioactive polypeptide which can be used in accordance with the invention or a precursor. The polypeptide can be encoded by a full-length sequence or by any part of the encoding sequence as long as the specific, for example ion-channel, activity is retained.

It is known that small changes in the sequence of the nucleic acids which can be used in accordance with the invention can be present, for example as a result of the degeneracy of the genetic code, or that untranslated sequences can be appended at the 5′ end and/or 3′ end of the nucleic acid, without the activity of the latter being significantly altered. This invention therefore also includes what are termed ‘variants’ of the above-described nucleic acids.

‘Stringent hybridization conditions’ are to be understood as meaning the conditions under which a hybridization takes place at 60° C. in 2.5×SSC buffer, followed by several washing steps at 37° C. in a lower buffer concentration, such as 0.5×SSC buffer, and is stable.

The term ‘variants’ designates any DNA sequences which are complementary to a DNA sequence, which hybridize with the reference sequence under stringent conditions and encode a polypeptide which exhibits an activity which is similar to that of the polypeptide encoded by the reference sequence.

Variants of the nucleic acids can also be part of the nucleic acids used in accordance with the invention having a length of at least 8 nucleotides, preferably having a length of at least 18 nucleotides, in particular having a length of at least 24 nucleotides.

The nucleic acids which are used in accordance with the invention are preferably DNA or RNA, preferably a DNA, in particular a double-stranded DNA. Furthermore, the sequence of the nucleic acids can be characterized in that it contains at least one intron and/or one polyA sequence. The nucleic acids which are used in accordance with the invention can also be employed in the form of their antisense sequence.

It is furthermore possible to use a nucleic acid which has been prepared synthetically for implementing the invention. Thus, the nucleic acid which can be used in accordance with the invention can, for example, be synthesized chemically, for example by the phosphotriester method, using the cDNA sequences depicted in SEQ ID No. 1 or SEQ ID No. 2 and/or using the protein sequences depicted in SEQ ID No. 3 or SEQ ID No. 4 (see also Table 1) while referring to the genetic code (see, e.g., Uhlmann, E. & Peyman, A. (1990) Chemical Reviews, 90, 543-584, No. 4).

Another use of the nucleic acid sequences which are employed in accordance with the invention is that of constructing antisense oligonucleotides (Zheng and Kemeny, 1995, Clin. Exp. Immunol. 100: 380-2; Nellen and Lichtenstein, 1993, Trends Biochem. Sci. 18: 419-23; Stein, 1992, Leukemia 6: 967-74) and/or ribozymes (Amarzguioui, et al. 1998, Cell. Mol. Life Sci. 54: 1175-202; Vaish, et al., 1998, Nucleic Acids Res. 26: 5237-42; Persidis, 1997, Nat. Biotechnol. 15: 921-2; Couture and Stinchcomb, 1996, Trends Genet. 12: 510-5) and/or what are termed small interfering RNA molecules (siRNAs) (Elbashir et al., Nature 2001; 411: 494-8). Antisense oligonucleotides can be used to decrease the stability of the above-described nucleic acid and/or inhibit the translation of the above-described nucleic acid. A similar approach is that of using siRNA oligonucleotides, which likewise lead to a decrease in the quantity of polypeptide. Thus, the expression of the corresponding genes in skin cells can, for example, be decreased both in vivo and in vitro by using antisense molecules and/or siRNA molecules and/or ribozymes. These oligonucleotides can therefore be suitable for use as a therapeutic agent. This strategy is particularly suitable for skin cells, preferably for keratinocytes, when the antisense oligonucleotides are complexed with liposomes (Smyth et al., 1997, J. Invest. Dermatol. 108: 523-6; White et al., 1999, J. Invest. Dermatol. 112: 699-705; White et al., 1999, J. Invest. Dermatol. 112: 887-92). A single-stranded DNA or RNA is preferred for use as a probe or as an antisense oligonucleotide.

The probe according to the invention can be used for diagnosing diseases which are connected with disturbed keratinocyte activity, where appropriate in combination or together with suitable additives and/or auxiliary substances.

As a rule, oligonucleotides are rapidly broken down by endonucleases or exonucleases, in particular by DNases and RNases which are present in the cell. For this reason, is it advantageous to modify the nucleic acid in order to stabilize it against breakdown such that a high concentration of the nucleic acid is maintained in the cell over a long period (Beigelman et al., 1995, Nucleic Acids Res. 23: 3989-94; Dudycz, 1995, WO 95/11910; Macadam et al., 1998, WO 98/37240; Reese et al., 1997, WO 97/29116). Typically, such a stabilization can be obtained by introducing one or more internucleotide phosphorus groups or by introducing one or more non-phosphorus internucleotides.

Uhlmann and Peymann (1990 Chem. Rev. 90, 544) provide a summary of suitable modified internucleotides (see also Beigelman et al., 1995 Nucleic Acids Res. 23: 3989-94; Dudycz, 1995, WO 95/11910; Macadam et al., 1998, WO 98/37240; Reese et al., 1997, WO 97/29116). Modified internucleotide phosphate radicals and/or non-phosphorus bridges in a nucleic acid which can be employed in one of the uses according to the invention contain, for example, methylphosphonate, phosphorothioate, phosphoramidate, phosphorodithioate or phosphate ester, whereas non-phosphorus internucleotide analogs contain, for example, siloxane bridges, carbonate bridges, carboxymethyl esters, acetamidate bridges and/or thioether bridges. The intention also is that this modification should improve the durability of a pharmaceutical composition which can be employed in one of the uses according to the invention.

In another embodiment of the invention, the nucleic acids which can be used in accordance with the invention are employed for preparing a vector, preferably in the form of a shuttle vector, phagemid, cosmid, expression vector or vector which is effective in gene therapy. In addition, the above-described nucleic acids can be used to prepare knock-out gene constructs or expression cassettes.

Thus, the nucleic acid which can be used in accordance with the invention can be present in a vector, preferably in an expression vector or a vector which is effective in gene therapy. The vector which is effective in gene therapy preferably contains skin-specific or keratinocyte-specific regulatory sequences which are functionally linked to the above-described nucleic acid.

In general, preference is given to using a double-stranded DNA for expressing the relevant gene, with particular preference being given to the DNA region which encodes the polypeptide. This region begins, for example, with the first start codon (ATG) which is located in a Kozak sequence (Kozak, 1987, Nucleic Acids Res. 15: 8125-48) up to the next stop codon (TAG, TGA or TAA) which is located in the same reading frame as the ATG. In prokaryotes, this region begins with a conserved sequence of 6 nucleotides, i.e. what is termed the Shine-Dalgarno sequence (Shine & Dalgarno, 1975, Eur J. Biochem. 57: 221-30), which is located a few nucleotides upstream of the start codon.

The expression vectors can be prokaryotic or eukaryotic expression vectors. Examples of prokaryotic expression vectors are, e.g., the vectors pGEM or pUC derivatives, for expression in E. coli, while examples of eukaryotic expression vectors are, e.g., the vectors p426Met25 and p426GAL1, for expression in Saccharomyces cerevisiae (Mumberg et al. (1994) Nucl. Acids Res., 22, 5767-5768), e.g., baculovirus vectors, for expression in insect cells, as disclosed in EP-B1-0 127 839 or EP-B1-0 549 721, and, e.g., the vectors Rc/CMV and Rc/RSV or SV40 vectors, for expression in mammalian cells, all of which can be obtained generally.

In general, the expression vectors also contain promoters which are suitable for the given host cell, such as the trp promoter for expression in E. coli (see, e.g., EP-B1-0 154 133), the Met 25, GAL 1 or ADH2 promoter for expression in yeasts (Russel et al. (1983), J. Biol. Chem. 258, 2674-2682; Mumberg, see above) or the baculovirus polyhedrin promoter for expression in insect cells (see z.13 EP-B1-0 127 839). Regulatory elements which permit constitutive, regulable, tissue-specific, cell cycle-specific or metabolism-specific expression in eukaryotic cells are suitable, for example, for expression in mammalian cells. Regulatory elements according to the present invention contain promoter, activator, enhancer, silencer and/or repressor sequences.

Examples of suitable regulatory elements which permit constitutive expression in eukaryotes are promoters which are recognized by RNA polymerase III or viral promoters, CMV enhancer, CMV promoter, SV40 promoter or LTR promoters, e.g. derived from MMTV (mouse mammary tumor virus; Lee et al. (1981) Nature 214, 228-232) and other viral promoter and activator sequences which are derived, for example, from HBV, HCV, HSV, HPV, EBV, HTLV or HIV.

Examples of regulatory elements which permit inducible expression in eukaryotes are the tetracycline operator in combination with an appropriate repressor (Gossen M. et al. (1994) Curr. Opin. Biotechnol. 5, 516-20).

Expression of the genes which are used in accordance with the invention preferably takes place under the control of tissue-specific promoters, with skin-specific promoters, such as the human K10 promoter (Bailleul et al., 1990. Cell 62: 697-708), the human K14 promoter (Vassar et al., 1989, Proc. Natl. Acad. Sci. USA 86: 1563-67) or the bovine cytokeratin IV promoter (Fuchs et al., 1988; The biology of wool and hair (eds.: G. E. Rogers, et al.), pp. 287-309. Chapman and Hall, London/New York) being particularly to be preferred.

Other examples of regulatory elements which permit tissue-specific expression in eukaryotes are promoters or activator sequences derived from promoters or enhancers belonging to those genes which encode proteins which are only expressed in particular cell types, preferably keratinocytes.

Examples of regulatory elements which permit cell cycle-specific expression in eukaryotes are promoters of the following genes: cdc25, cyclin A, cyclin E, cdc2, E2F, B-myb and DHFR (Zwicker J. and Müller R. (1997) Trends Genet. 13, 3-6).

Examples of regulatory elements which permit metabolism-specific expression in eukaryotes are promoters which are regulated by hypoxia, by glucose lack, by phosphate concentration or by heat shock.

Examples of regulatory elements which simultaneously permit expression which is spatially and temporally restricted are nucleic acids which encode a fusion protein, with the nucleic acid being present between the sequence for the site-specific recombinase Cre and a modified estrogen receptor which is under the control of a tissue-specific promoter. The resulting, tissue-specific cytoplasmic fusion protein can translocate into the cell nucleus, as a result of administering the estrogen analog tamoxifen, and generate recombinations which lead to a change in gene expression (Feil et al., 1996, Proc Natl Acad Sci 93: 10887-90). In order to enable the nucleic acids which can be used in accordance with the invention to be introduced by means of transfection, transformation or infection, and thereby permit expression of the polypeptide in a eukaryotic or prokaryotic cell, the nucleic acid can be present as a plasmid, as part of a viral or nonviral vector. Viral vectors which are particularly suitable in this context are: baculoviruses, vaccinia viruses, adenoviruses, adeno-associated viruses and herpesviruses. Particularly suitable nonviral vectors in this context are: virosomes, liposomes, cationic lipids and polylysine-conjugated DNA.

Examples of vectors which are effective in gene therapy are viral vectors, for example adenoviral vectors or retroviral vectors (Lindemann et al., 1997, Mol. Med. 3: 466-76; Springer et al., 1998, Mol. Cell. 2: 549-58). Eukaryotic expression vectors are suitable for use in gene therapy in isolated form since, when applied topically, naked DNA is able to penetrate into skin cells (Hengge et al., 1996, J. Clin. Invest. 97: 2911-6; Yu et al., 1999, J. Invest. Dermatol. 112: 370-5).

Vectors which are effective in gene therapy can also be obtained by complexing the nucleic acid which is used in accordance with the invention with liposomes since it is possible, in this way, to achieve a very high efficiency of transfection, particularly of skin cells (Alexander and Akhurst, 1995, Hum. Mol. Genet. 4: 2279-85). In lipofection, small unilamellar vesicles composed of cationic lipids are prepared by ultrasonicating the liposome suspension. The DNA is bonded ionically on the surface of the liposomes in a ratio which is such that a positive net charge remains and the plasmid DNA is completely complexed by the liposomes. In addition to the DOTMA (1,2-dioleyloxypropyl-3-trimethylammonium bromide) and DPOE (dioleoylphosphatidylethanolamine) lipid mixtures employed by Felgner et al. (1987, see above), a large number of new lipid formulations have by now been synthesized and tested for their efficiency in transfecting various cell lines (Behr, J. P. et al. (1989), Proc. Natl. Acad. Sci. USA 86, 6982-6986; Felgner, J. H. et al. (1994) J. Biol. Chem. 269, 2550-2561; Gao, X. & Huang, L. (1991), Biochim. Biophys. Acta 1189, 195-203). Examples of the new lipid formulations are DOTAP N-[1-(2,3-dioleoyloxy)propyl]-N,N,N-trimethylammonium ethyl sulfate and DOGS (TRANSFECTAM; dioctadecylamidoglycylspermine).

Auxiliary substances which increase the transfer of nucleic acids into the cell can, for example, be proteins or peptides which are bonded to DNA or synthetic peptide-DNA molecules which enable the nucleic acid to be transported into the nucleus of the cell (Schwartz et al. (1999) Gene Therapy 6, 282; Branden et al. (1999) Nature Biotech. 17, 784). Auxiliary substances also include molecules which enable nucleic acids to be released into the cytoplasm of the cell (Planck et al. (1994) J. Biol. Chem. 269, 12918; Kichler et al. (1997) Bioconj. Chem. 8, 213) or, for example, liposomes (Uhlmann and Peymann (1990) see above). Another, particularly suitable form of gene-therapy vector can be obtained by applying the nucleic acids which are used in accordance with the invention to gold particles and shooting these particles into tissues, preferably into the skin, or cells using what is termed the gene gun (Wang et al., 1999, J. Invest. Dermatol., 112: 775-81, Tuting et al., 1998, J. Invest. Dermatol. 111: 183-8). When the skin is bombarded, epidermal cells and keratinocytes, which constitute the main constituent of the epidermis, are transfected preferentially (Udvardi et al., 1999, J. Mol. Med. 77: 744-750; Lu and Goldsmith, 1996, Proc. Assoc. Am. Physicians, 108: 165-172).

Another form of a vector which is effective in gene therapy can be prepared by introducing ‘naked’ expression vectors into a biocompatible matrix, for example a collagen matrix. This matrix can be introduced into skin in which keratinocyte activity is disturbed, for example into a wound, in order to transfect the immigrating cells with the expression vector and to express the polypeptides which are used in accordance with the invention in the cells (Goldstein and Banadio, U.S. Pat. No. 5,962,427).

It is also advantageous, for using the above-described nucleic acid in gene therapy, if the part of the nucleic acid which encodes the polypeptide contains one or more noncoding sequences, including intron sequences, preferably between the promoter and the start codon of the polypeptide, and/or a polyA sequence, in particular the naturally occurring polyA sequence or an SV40 virus polyA sequence, especially at the 3′ end of the gene, since this makes it possible to stabilize the mRNA (Jackson, R. J. (1993) Cell 74, 9-14 and Palmiter, R. D. et al. (1991) Proc. Natl. Acad. Sci. USA 88, 478-482).

The present invention also relates to a host cell, preferably a skin cell, in particular a keratinocyte, which [lacuna] at least one vector and/or at least one knock-out gene construct which includes a nucleic acid, or variants thereof, which encodes a polypeptide, or functional variants thereof, as depicted in SEQ ID No.

ID No. 3 or SEQ ID No. ID No. 4. The skilled person is familiar with knock-out gene constructs, for example from US patents U.S. Pat. Nos. 5,625,122; 5,698,765; 5,583,278 and 5,750,825.

Host cells can be either prokaryotic or eukaryotic cells; examples of prokaryotic host cells are E. coli, and examples of eukaryotic cells are S. cerevisiae and insect cells.

The host cells according to the invention can be used for diagnosing, preventing and/or treating diseases which are connected with disturbed keratinocyte activity, in particular wound healing disturbances and/or psoriasis, or for identifying pharmacologically active substances.

A further preferred transformed host cell is a transgenic, embryonic nonhuman stem cell which contains at least one nucleic acid, or variant thereof, which encodes a polypeptide, or functional variant thereof, as depicted in SEQ ID No. ID No. 3 or SEQ ID No. ID No. 4. In a preferred embodiment, the above nucleic acid is contained in a knock-out gene construct or an expression cassette or an above-described stem cell. Methods for transforming. Transfecting or infecting host cells and/or stem cells are well known to the skilled person and include, for example, electroporation and microinjection.

The invention also relates to a transgenic, nonhuman mammal which contains at least one above-described knock-out gene construct or an above-described expression cassette, preferably integrated into the genome. In general, transgenic animals exhibit an expression of the nucleic acids and/or polypeptides which is elevated in a tissue-specific manner and can be used for analyzing and/or diagnosing diseases which are connected with disturbed keratinocyte activity, in particular wound healing disturbances and/or psoriasis. Thus, for example, an activin A-transgenic mouse exhibits improved wound healing (Munz et al., 1999, EMBO J. 18: 5205-15) whereas a transgenic mouse containing a dominantly negative KGF receptor exhibits delayed wound healing (Werner et al., 1994, Science 266: 819-22).

Methods for preparing transgenic animals, in particular the mouse, are likewise known to the skilled person from DE 196 25 049 and the patents U.S. Pat. Nos. 4,736,866; 5,625,122; 5,698,765; 5,583,278 and 5,750,825 and involve transgenic animals which can be generated, for example, by directly injecting expression vectors (see above) into embryos or spermatocytes, by transfecting expression vectors into embryonic stem cells (Polites and Pinkert: DNA Microinjection and Transgenic Animal Production, pages 15 to 68 in Pinkert, 1994: Transgenic animal technology: a laboratory handbook, Academic Press, London, UK; Houdebine, 1997, Harwood Academic Publishers, Amsterdam, The Netherlands; Doetschman: Gene Transfer in Embryonic Stem Cells, pages 115 to 146 in Pinkert, 1994, see above; Wood: Rebrovirus-Mediated Gene Transfer, pages 147 to 176 in Pinkert, 1994, see above; Monastersky: Gene Transfer Technology: Alternative Techniques and Applications, pages 177 to 220 in Pinkert, 1994, see above) or by isolating cell nuclei from differentiated somatic cells, for example fibroblasts, and then transferring them into denucleated oocytes (McCreath et al., 2000, Nature 405: 1004-5). Thus, known knock-out mouse models, such as the eNOS (Lee et al., 1999, Am. J. Physiol. 277: H1600-H1608), Nf-1 (Atit et al., 1999, J. Invest. Dermatol. 112: 835-42) and osteopontin (Liaw et al., 1998, J. Clin. Invest. 101: 967-71) knock-out mice, exhibit impaired wound healing.

In the same way, nucleic acids which can be used in accordance with the invention can [lacuna] integrated into what are termed targeting vectors (Pinkert, 1994, see above) [lacuna] it is possible, after transfecting embryonic stem cells and homologous recombination, to generate, for example, knock-out mice which in general, as heterozygous mice, exhibit a decreased expression of nucleic acid whereas homozygous mice no longer exhibit any expression of the nucleic acid. Tissue-specific reduction in the expression of genes which are used in accordance with the invention, for example in skin-specific cells, in particular in keratinocytes using the Cre-loxP system (stat3 knock-out, Sano et al., EMBO J. 1999 18: 4657-68), is particularly to be preferred in this connection. The transgenic and knock-out cells or animals which are generated in this way can be used, for example, for analyzing diseases which are connected with disturbed keratinocyte activity, for example a wound. However, they can also be used for screening and identifying pharmacologically active substances or vectors which are active in gene therapy.

The invention also relates to a process for preparing a polypeptide for diagnosing and/or preventing and/or treating diseases which are connected with disturbed keratinocyte activity, in particular wound healing disturbances and/or psoriasis, or for identifying pharmacologically active substances in a suitable host cell, which process is characterized in that an above-described nucleic acid is used.

The polypeptide is prepared, for example, by using methods which are well known to the skilled person to express the above-described nucleic acid in a suitable expression system, as already explained above. Examples of suitable host cells are the E. coli strains DHS, HB101 or BL21, the yeast strain S. cerevisiae, the insect cell line lepidopteran, e.g. from Spodoptera frugiperda, or the animal cells COS, Vero, 293, HaCaT and HeLa, all of which are available generally.

The invention also relates to a process for preparing a fusion protein for diagnosing and/or preventing and/or treating diseases which are connected with disturbed keratinocyte activity or for identifying pharmacologically active substances in a suitable host cell, which process uses an above-described nucleic acid.

In another embodiment of the invention, a functional variant of the polypeptides which can be used in accordance with the invention is a fusion protein, i.e. fusion proteins containing at least one polypeptide as depicted in SEQ ID No. 3 or SEQ ID No. 4 or a functional variant thereof. These fusion proteins can be used for diagnosing, preventing and/or treating diseases which are connected with disturbed keratinocyte activity, in particular wound healing disturbances and/or psoriasis, or for identifying pharmacologically active substances.

Fusion proteins are prepared in this connection which contain the above-described polypeptides, with the fusion proteins either themselves already exhibiting the function of an above-described polypeptide or only being functionally active the specific function after the fusion moiety has been eliminated. In particular, these fusion proteins include fusion proteins which contain a moiety of approx. 1-300, preferably approx. 1-200, particularly preferably approx. 1-150, in particular approx. 1-100, especially approx. 1-50, foreign amino acids. Examples of such peptide sequences are prokaryotic peptide sequences which can be derived, for example, from E. coli galactosidase. In addition, it is also possible to use viral peptide sequences, for example from the bacteriophage M13, in order, in this way, to generate fusion proteins for the phage-display method, with which the skilled person is familiar.

Other preferred examples of peptide sequences for fusion proteins are peptides which facilitate detection of the fusion protein; these include, for example, green fluorescent protein or variants thereof.

In order to purify the above-described proteins, it is possible to attach an additional polypeptide (tag). Suitable protein tags make it possible, for example, to achieve high-affinity absorption to a matrix, stringent washing with suitable buffers, without the complex being eluted to any significant degree, and subsequent selective elution of the absorbed complex. Examples of protein tags which are known to the skilled person are the (His)₆ tag, the Myc tag, the FLAG tag, the hemagluteinin tag, the glutathione transferase (GST) tag, intein containing an affinity chintin-binding tag or the maltose-binding protein (MBP) tag. These protein tags can be located N-terminally, C-terminally and/or within the protein.

The invention also relates to a process for preparing an antibody, preferably a polyclonal or monoclonal antibody, for diagnosing, preventing and/or treating diseases which are connected with disturbed keratinocyte activity, in particular wound healing disturbances and/or psoriasis, or for identifying pharmacologically active substances, in which process a polypeptide, or functional equivalents thereof, or parts thereof containing at least 6 amino acids, preferably containing at least 8 amino acids, in particular containing at least 12 amino acids, is/are used in accordance with the present invention.

The process is effected, using methods which are well known to the skilled person, by immunizing a mammal, for example a rabbit, with the above-described polypeptide, or the said parts thereof, where appropriate in the presence of, e.g., Freund's adjuvant and/or aluminum hydroxide gels (see, e.g., Diamond, B. A. et al (1981) The New England Journal of Medicine, 1344-1349). The polyclonal antibodies which are formed in the animal as a result of an immunological reaction can then be readily isolated from the blood, and purified, for example by means of column chromatography, using well known methods. Monoclonal antibodies can be prepared, for example, using the known method of Winter & Milstein (Winter, G. & Milstein, C. (1991) Nature, 349, 293-299).

The present invention also relates to an antibody for analyzing, diagnosing, preventing and/or treating diseases which are connected with disturbed keratinocyte activity, in particular wound healing disturbances and/or psoriasis, or for identifying pharmacologically active substances which are directed against an above-described polypeptide, or functional variants thereof, and react specifically with the above-described polypeptides, with the above-mentioned parts of the polypeptide either themselves being immunogenic or being able to be made immunogenic, or to have their immunogenicity increased, by being coupled to suitable carriers, such as bovine serum albumin. This antibody is either polyclonal or monoclonal; it is preferably a monoclonal antibody. According to the present invention, the term antibody is also understood as meaning recombinantly prepared and optionally modified antibodies or antigen-binding parts thereof, such as chimeric antibodies, humanized antibodies, multifunctional antibodies, bispecific antibodies, oligospecific antibodies, single-stranded antibodies, F(ab) fragments or F(ab)₂ fragments (see, e.g., EP-B1-0 368 684, U.S. Pat. Nos. 4,816,567, 4,816,397, WO 88/01649, WO 93/06213, WO 98/24884).

The invention also relates to the use of an antibody which is directed against a polypeptide, or functional variants thereof, as depicted in SEQ ID No. ID No. 3 or SEQ ID No. ID No. 4 for diagnosing and/or preventing and/or treating diseases which are connected with disturbed keratinocyte activity, in particular wound healing disturbances and/or psoriasis, particularly preferably psoriasis.

Thus, the local injection of monoclonal antibodies directed against TGF-β1 can improve wound healing in an animal model (Ernst et al., 1996, Gut 39: 172-5).

The invention also relates to the use of modulators of the activity of a polypeptide, or functional variants thereof, as depicted in SEQ ID No. 3 or SEQ ID No. 4, preferably of a polypeptide as depicted in SEQ ID No. 3 or SEQ ID No. 4, for diagnosing and/or preventing and/or treating diseases which are connected with disturbed keratinocyte activity.

Within the meaning of the invention, the term ‘modulator’ encompasses chemical substances, compounds, compositions and mixtures which exert an influence on the the activity of the ion channel IK according to the invention in that the modulator either opens it (=activates) or closes it (=inhibits). Furthermore, the modulator can modulate the kinetics of the channel activity. A modulator can be an inhibitor which inhibits the activity of the ion channel or an activator which activates the activity of the ion channel.

In addition, the term “modulatOR” also encompasses compounds, compositions and mixtures which modulate the expression of the polypeptides as depicted in SEQ ID No. 3 or SEQ ID No. 4 or functional variants thereof. The modulation can take place, for example, at the transcriptional or translational level. Substances which increase the quantity of the polypeptides which can be used in accordance with the invention are activators whereas substances which reduce the quantity of the polypeptides which can be used in accordance with the invention are inhibitors.

Methods for determining channel activity are known to the skilled person from the literature. For example, IK channels have been expressed in oocytes by means of in-vitro transcription and injection, and the ion channel activity has been measured using the patch-clamp method (WO 98/11139; Gerlach et al., 2000, J. Biol. Chem., 275: 585-598; Khanna et al., 1999, J. Biol. Chem., 274: 14838-14849). Furthermore, IK channels can be expressed in human cell lines and analyzed using the patch-clamp method (WO 00/37422; WO 99/25347). It is also possible to investigate cells possessing endogenously expressed IK channels in this way (Gerlach et al., 2000, J. Biol. Chem., 275: 585-598). IK channels which are expressed endogenously, or by means of transfection, in skin cells, for example keratinocytes, can be used in an analogous manner. Examples of other methods which are suitable for identifying modulators of channels are radioactive Ru+flux test systems and fluorescence test systems using voltage-dependent dyes (Vestergaard-Bogind et al., 1988, J. Membr. Biol. 88: 67-75; Daniel et al., 1991, J. Pharmacol. Meth. 25: 185-193; Holevinsky et al. 1994, J. Membr. Biol. 137: 59-70). For example, it is possible to use an FLIPR (fluorescence image plate reader) test system (WO 99/25347). Test systems for testing the inhibitory or activating effect of a substance on the potassium flow through an IK channel can be implemented by adding the substances to a solution which is in contact with IK channel-expressing cells (e.g. Blatz et al., 1986, Nature, 323: 718-720; Park, 1994, J. Physiol., 481: 555-570).

In general, the activity of a channel can be determined by measuring the electric current or the ion flow or the secondary effects of the electric current or of the ion flow. Modulators of the activity of the ion channel alter the electric current or the ion flow. In this way, it is possible to study the modulatory properties of substances which are to be tested.

Changes in the cation flow through the channel can be detected, for example, either directly by determining the changes in the concentrations of the ions or indirectly by measuring the membrane potential by means of radioactively labeling the ions. If the influence of the modulators on intact cells or organisms is being detected, it is possible to measure a very wide variety of secondary physiological effects. Possible effects are transcriptional changes, changes in cell volume, changes in the metabolism of the cell, the release of hormones, etc. (WO 98/11139). Wo 99/25347 discloses methods for screening a chemical substance for a modulatory effect on an IK channel.

None of the known modulators of the polypeptides as depicted in SEQ ID No. 3 or SEQ ID No. 4 has previously been reported to have a connection with diseases which are connected with disturbed keratinocyte activity, in particular wound healing disturbances and/or psoriasis, for example in association with disturbed wound healing, nor has such a connection been suggested. It was therefore unexpected that these compounds can be used in accordance with the invention.

In another embodiment of the invention, the modulator is an inhibitor.

According to the invention, it is possible, for the purpose of modulating the IK channels, to use at least one antibody which is directed against a polypeptide, or functional variant thereof, as depicted in SEQ ID No. ID No. 3 or SEQ ID No. ID No. 4. In this connection, the antibodies can have an inhibitory effect or an activating effect on the activity of the polypeptide which can be used in accordance with the invention.

Inhibitors of IK channel activity have been disclosed, for example, in WO 99/25347. For example, it is possible to use a symmetric or asymmetric derivative of 1,4-dihydropyridine-3,5-dicarboxylic acid in accordance with the general formula (I):

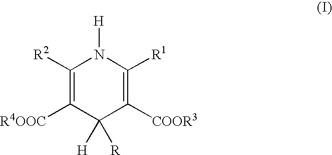

or a pharmaceutically tolerated salt, an oxide or a hydrate thereof;

where

R is an alkyl group or cycloalkyl group which is optionally substituted by halogen, preferably by F or Cl;

or R is a monocyclic or polycyclic aryl group, where the aryl group can be substituted, once or more than once, by a substituent selected from halogen, in particular F or Cl, trifluoromethyl (—CF₃), nitro (—NO₂), cyano (—CN), azido (—N₃), a group of the formula —S(O)ₙ-alkyl, —S(O)ₙ—NH-alkyl or —S(O)—N-(alkyl)₂, where n can preferably be 0, 1 or 2, an alkyl group, a cycloalkyl group, an alkoxy group, a trifluoromethyloxy group (—OCF₃), a carboxyl group (—COOH), a group of the formula —COO-alkyl, a carbamoyl group (—CONH₂), and a group of the formula —CONH-alkyl or CON(alkyl)₂;

or R is a monoheterocyclic or polyheterocyclic group, where the heterocyclic group can be substituted, once or more than once, by an alkyl group, an alkoxy group, a carboxyl group (—COOH), a group of the formula —COO—alkyl, and/or a group of the formula —COO-phenyl, with preferred heteroatoms being N, S and O;

and R¹, R², R³ and R⁴ are, independently of each other, hydrogen, an alkyl group, a cycloalkyl group, an alkenyl group, an alkynyl group, an alkoxy group, a phenyl group, a phenylalkyl group, a furanyl group, a furanylalkyl group, a pyridyl group or a pyridylalkyl group which is optionally substituted by halogen, preferably by F or CL.

A preferred embodiment is that of using an inhibitor of the general formula (I) in which R is a C₃₋₇ cycloalkyl group, in particular a cyclohexyl group; or a phenyl group, preferably a monosubstituted phenyl group, where the phenyl group [lacuna] once or more than once by a substituent selected from the group consisting of halogen, trifluoromethyl (—CF₃), nitro (—NO₂) and cyano (—CN);

or R is a pyridyl group or a dihydropyridyl group, where this group can be monosubstituted by a group of the formula —COO-alkyl or a group of the formula —COO—phenyl.

In another preferred embodiment, it is possible to use an inhibitor according to the general formula (I) where R is a 2-nitrophenyl group, a 3-nitrophenyl group, a 4-nitrophenyl group, a 2-trifluoromethylphenyl group, a 3-trifluoromethylphenyl group, a 4-trifluoroethylphenyl group, a 2-cyanophenyl group, a 3-cyanophenyl group or a 4-cyanophenyl group;

or R is a 2-pyridyl, 4-pyridyl, 3-pyridyl, a 1,2- or 1,4- or 1,6-dihydro-2-pyridyl group, a 1,2- or 1,4- or 1,6-dihydro-3-pyridyl group or a 1,2- or 1,4- or 1,6-dihydro-4-pyridyl group, where the pyridyl or dihydropyridyl groups can be monosubstituted by C₁₋₆-alkyl, a group of the formula —COO—C₁₋₆-alkyl or a group of the formula —COO-phenyl.

In another preferred embodiment, use is made of a modulator of the general formula (I) where R¹, R², R³, and R⁴ are, independently of each other, C₁₋₆-alkyl, preferably methyl, ethyl, propyl, isopropyl, butyl or isobutyl.

A preferred embodiment is that of using an inhibitor of the general formula (I) where the inhibitor is an asymmetric derivative of 1,4-dihydropyridine-3,5-dicarboxylic acid in accordance with general formula (I). Preference is given to asymmetric derivatives which are asymmetric C₁₋₆-alkyl derivatives of 1,4-dihydropyridine-3,5-dicarboxylic acid (I). For example, it is possible to use the following substances:

ethylmethyl 1,4-dihydro-2,6-dimethyl-4-(2-nitrophenyl)-pyridine-3,5-dicarboxylate;

propylmethyl 1,4-dihydro-2,6-dimethyl-4-(2-nitro-phenyl)-pyridine-3,5-dicarboxylate;

ethylmethyl 1,4-dihydro-2,6-dimethyl-4-(3-nitrophenyl)-pyridine-3,5-dicarboxylate.

Another preferred embodiment is that of using a symmetric derivative of 1,4-dihydropyridine-3,5-dicarboxylic acid depicted by the general formula (I).

Preferred compounds contain symmetric C₁₋₆-alkyl derivatives of 1,4-dihydropyridine-3,5-dicarboxylic acid. For example, it is possible to use the following substances in accordance with the invention:

dimethyl 1,4-dihydro-2,6-dimethyl-4-(2-nitrophenyl)-pyridine-3,5-dicarboxylate (nifedipine);

diethyl 1,4-dihydro-2,6-dimethyl-4-(2-nitrophenyl)-pyridine-3,5-dicarboxylate.

In another embodiment, it is possible to use, in accordance with the invention, inhibitors which are derivatives or metabolites of clotrimazole in accordance with the general formula (II) (see also WO 99/25347)

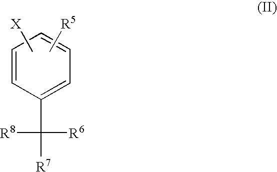

or a pharmaceutically tolerated salt, an oxide or a hydrate thereof;

where

X is halogen, in particular F or Cl, a trifluoromethyl group, a nitro group or a cyano group;

R⁵ is hydrogen, halogen, in particular F or Cl, hydroxyl, an alkyl group, a cycloalkyl group, an alkoxy group or an alkyloxy group, where appropriate substituted by halogen, preferably F or Cl;

R⁶ is hydrogen or a phenyl group, where the phenyl group can be substituted, once or more than once, by substituents selected from halogen, preferably F or Cl, and hydroxyl;

R⁷ is hydrogen, halogen, preferably F or Cl, hydroxyl, alkyl or alkoxy, where appropriate substituted by halogen, preferably F or Cl;

R⁸ is a group of the formula —Y—CH₂—R⁹, where Y is oxygen (—O—) or sulfur (—S—); a group of the formula ═NO—CH₂—R⁹; a group of the formula —O-phenyl-CH═CH₂; a group of the formula —CH₂—CH(CH₃)—S-phenyl, where the phenyl group can be substituted, once or more than once, by substituents selected from halogen, preferably F or Cl, and hydroxyl; or a phenyl group, where the phenyl group can be substituted, once or more than once, by substituents selected from halogen, preferably F or Cl, and hydroxyl; and where R⁹ is an ethenyl group (CH₂═CH—); a phenyl group, where the phenyl group can be substituted, once or more than once, by substituents selected from halogen, preferably F or Cl, and hydroxyl group; a phenyl-S-phenyl group; a group of the formula CH₂—O-phenyl, where the phenyl group can be substituted, once or more than once, by substituents selected from halogen, preferably F or Cl, and hydroxyl group; or a group of the general formula (VII),

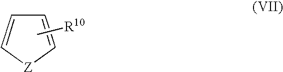

where Z is S, O or N;

and R¹⁰ is hydrogen or halogen, preferably F or Cl, or hydroxyl group.

Preference is given to using the following derivatives and metabolites:

2-chlorophenyl-4-hydroxyphenylphenylmethane; 2-chloro-phenylbisphenylmethane; 2-chlorophenylbisphenylmeth-anol; 3-(1-[2,4-dichlorophenyl]ethoxymethyl)-2-chloro-thiophene; O-(2,4-dichlorobenzyl)-2,4-dichloroaceto-phenoneoxime; 1-(2,4-dichloro)-1-(4-(phenylthio)benzyl-oxy)ethane; 1-(2,4-dichlorophenyl)-1-1-(allyloxy)-ethane; 1-(2,4-dichlorophenyl)-1-(4-chlorobenzylthio)-ethane; 1-(2,4-dichlorophenyl)-1-(2,4-dichlorobenzyl-oxy)ethane; 1-(2,4-dichlorophenyl)ethyl-2,6-dichloro-benzyl ether; 1-(2-[4-chlorophenoxy]ethyloxy)-1-(2,4-dichlorophenyl)propene; 1-(2,4-dichlorophenyl)ethyl-(4-chlorophenyl)methyl ether; 3-chlorobenzyl-2-vinylphenyl ether and 1-(4-chlorophenyl)-3-(2,6-dichlorophenylthio)butane.

The document WO 99/25347 discloses other substances which act as inhibitors of hIK1 and can be used in accordance with the invention. Examples are the imidazole derivatives miconazole, econazole, butoconazole, oxiconazole, sulconazole and thioconazole, the triazole derivatives fluconazole, terconazole and itraconazole, the nitroimidazole derivatives metronidazole, tinidazole, nimorazole, ornidazole and benznidazole.

Other suitable modulators which can be used in accordance with the invention are oxime derivatives in accordance with the general formula (III) (see WO 00/34228):

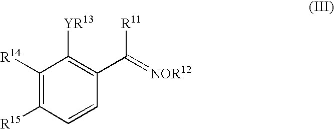

or a pharmaceutically tolerated salt, an oxide or a hydrate thereof;

where

Y is oxygen, sulfur or an amino group, preferably an amino group of the formula NHR¹³;

R¹¹ is hydrogen; an alkyl group; a cycloalkyl group; a hydroxyl group; an alkoxy group; an acyl group; a phenyl group or a benzyl group, where the group, in particular the phenyl group or benzyl group, can be substituted, once or more than once, by substituents selected from halogen, in particular F or Cl, —CF₃, —NO₂, —CN, alkyl group, cycloalkyl group, hydroxyl group and alkoxy group; a group of the formula CH₂CN; a group of the formula CH₂CO₂R′, where R′ is hydrogen or an alkyl group; a group of the formula CH₂CONR^{IV}R^{v}, where R^{IV} and R^{v} are, independently of each other, hydrogen or an alkyl group, where appropriate substituted by halogen; or a group of the formula —CH₂C (═NOH)NH₂;

R¹² is hydrogen; an alkyl group; a cycloalkyl group; a phenyl group or a benzyl group, where the group, in particular the phenyl group or benzyl group, can be substituted, once or more than once, by substituents selected from halogen, in particular F or Cl, —CF₃, —NO₂, —CN, alkyl group, cycloalkyl group, hydroxyl group and alkoxy group;

R¹³ is hydrogen; an alkyl group; a cycloalkyl group; a phenyl group or a benzyl group, where the group, in particular the phenyl group or benzyl group, can be substituted, once or more than once, by substituents selected from halogen, in particular F or Cl, —CF₃, —NO₂, —CN, alkyl group, cycloalkyl group, hydroxyl group and alkoxy group; and

R¹⁴ and R¹⁵ are, independently of each other, hydrogen; halogen, in particular F or Cl; —CF₃; —NO₂; —CN; an alkyl group, an alkoxy group; a phenyl group or a benzyl group, where the group, in particular the phenyl group or benzyl group, can be substituted, once or more than once, by substituents selected from halogen, in particular F or Cl, —CF³, —NO₂, —CN, alkyl, cycloalkyl, hydroxyl and alkoxy; or are a group of the formula—SO₂NR″R″′, where R″ and R″′ are, independently of each other, hydrogen or an alkyl group;

or R¹⁴ and R¹⁵ together form an additional 4-membered to 7-membered fused ring, where the fused ring can be aromatic or partially saturated and can be substituted, once or more than once, by substituents selected from halogen, in particular F or Cl, —CF₃, —NO₂, —CN and a group of the formula —SO₂NR″R″′, where R″ and R″′ are, independently of each other, hydrogen or an alkyl group.

In a preferred embodiment of the modulator which can be used in accordance with the invention, R¹¹ is hydrogen or an alkyl group. In another preferred embodiment, R¹² is hydrogen, an alkyl group or a benzyl group or phenyl group, where the where the phenyl group or benzyl group can be substituted, once or more than once, by substituents selected from halogen, —CF₃, —NO₂, —CN, alkyl, cycloalkyl, hydroxyl and alkoxy.

In another preferred embodiment, Y is oxygen or an amino group of the formula NHR¹³, where R¹³ here is hydrogen, alkyl, benzyl or acetyl.

In another preferred embodiment, R¹⁴ and R¹⁵ are, independently of each other, hydrogen, halogen, alkyl or alkoxy.

In another preferred embodiment, R¹⁴ and R¹⁵ together form an additional 6-membered fused ring, where the fused ring can be aromatic or partially saturated, where the fused ring can be substituted, once or more than once, by substituents selected from halogen, in particular F or Cl, —CF₃, —NO₂, —CN and a group of the formula —SO₂NR″R″′, where R″ and R″′ are, independently of each other, hydrogen or an alkyl group.

In a particularly preferred embodiment, it is possible to use the following oxime derivatives as modulators in accordance with the invention: 2-aldoximo-1-naphthol; 2-aldoximo-5,6-dimethylphenol; 2-aldoximo-5,6-dichlorophenol; O-benzyl-2-formyl-5,6-dichlorophenoloxime; O-methyl-2-formyl-5,6-dichloro-phenoloxime; 2-aldoximo-5,6,7-8-tetrahydro-1-naphthol; 1-hydroxy-2-acetonaphthoneoxime; 1-methoxy-2-aceto-naphthoneoxime; O-ethyl-1-methoxy-2-acetonaphthone-oxime; 1-ethoxy-2-acetonaphthoneoxime; 1-benzyloxy-2-acetonaphthoneoxime; 2-hydroxy-3,4-dimethylaceto-phenoneoxime; 2-methoxy-3,4-dimethylaceto-phenoneoxime; 2-hydroxy-3,4-dimethoxyacetophenoneoxime; 2,3,4-tri-methoxyacetophenoneoxime; 1-hydroxy-5, 6,7,8-tetrahydro-2-acetonaphthoneoxime; 1-methoxy-5,6,7,8-tetrahydro-2-acetonaphthoneoxime; 1-(2-propyloxy)-2-acetonaphthone-oxime; N-(2-acetylphenyl)acetamideoxime.

In another embodiment, it is possible to use chemical compounds in accordance with the general formula (IV) (see WO 00/34248)

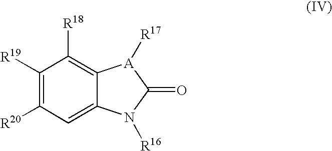

or a pharmaceutically tolerated salt, an oxide or a hydrate thereof, as modulators in accordance with the invention;

where

A is oxygen, sulfur or nitrogen;

R¹⁶ is hydrogen; an alkyl group; a cycloalkyl group; a hydroxyl group; an alkoxy group; an acyl group; a phenyl group or a benzyl group, where the group, in particular the phenyl group or benzyl group, can be substituted, once or more than once, by substituents selected from halogen, in particular F or Cl, —CF₃, —NO₂, —CN, alkyl, cycloalkyl, hydroxyl and alkoxy; a group of the formula CH₂CN; a group of the formula CH₂CO₂R′, where R′ is hydrogen or an alkyl group; a group of the formula CH₂CONR^{IV}R^{v}, where R^{IV} and R^{v} are, independently of each other, hydrogen or an alkyl group; or a group of the formula —CH₂C(═NOH)NH₂;

R¹⁷ is hydrogen; an alkyl group; a cycloalkyl group; a phenyl group or a benzyl group, where the group, in particular the phenyl group or benzyl group, can be substituted, once or more than once, by substituents selected from halogen, in particular F or Cl, —CF₃, —NO₂, —CN, alkyl, cycloalkyl, hydroxyl and alkoxy;

R¹⁸, R¹⁹ and R²⁰ are, independently of each other, hydrogen; halogen, in particular F or Cl; —CF₃; —NO₂; —CN; an alkyl group; an alkoxy group; a phenyl group or a benzyl group, where the group, in particular the phenyl group or benzyl group, can be substituted, once or more than once, by substituents selected from halogen, in particular F or Cl, —CF₃, —NO₂, —CN, alkyl, cycloalkyl, hydroxyl and alkoxy; or a group of the formula —SO₂NR″R′″, where R″ and R″′ are, independently of each other, hydrogen or an alkyl group, where appropriate substituted, preferably by F or Cl;

or R²⁰ is defined as above and R¹⁸ and R¹⁹ together form an additional 4-membered to 7-membered fused ring, where the fused ring can be heterocyclic, where the hetero atoms are preferably N, S or O, can be aromatic, saturated or partially saturated, and optionally [lacuna] by substituents selected from halogen, in particular F or Cl, —CF₃, —NO₂, —CN and a group of the formula —SO₂NR″R′″, where R″ and R″′ are, independently of each other, hydrogen or an alkyl group.

In a preferred embodiment, R¹⁶ is hydrogen or an alkyl group. In another preferred embodiment, R¹⁷ is hydrogen or an alkyl group. In another preferred embodiment, R¹⁹, R²⁰ and R¹⁸ are, independently of each other, hydrogen or an alkyl group.

Particular preference is given to the use, according to the invention, of 1-ethyl-4,5-dichlorobenzimidazolone, 1,3-diethyl-4,5-dichlorobenzimidazolone, 3-ethyl-5-chlorobenzoxazolone and the activators 1-ethyl-2-benzimidazolinone (1-EBIO), 1-ethyl-4,5-dichloro-2-benzimidazolinone (Boehringer et al., 2000 J. Med. Chem., 43: 2664), 5,6-dichloro-1-ethyl-1,3-dihydro-2H-benzimidazol-2-one (DCEBIO; Singh et al., 2001, J Pharmacol Exp Ther; 296: 600-11) and chlorzoxazone (Syme et al., 2000, Am. J. Physiol. 278: C570-C581).

In another preferred embodiment, use is made of a modulator in accordance with formula (IV) in which R¹⁸ and R¹⁹ together form a 5-membered or 6-membered fused ring, where the ring can be heterocyclic; in addition, the ring can be aromatic, saturated or partially saturated and the ring can be optionally substituted, once or more than once, by substituents selected from the group consisting of halogen, in particular F or Cl, —CF₃, —NO₂, —CN and a group of the formula —SO₂NR″R″′, where R″ and R″′ are, independently of each other, hydrogen or an alkyl group, where appropriate substituted, in particular by F or Cl.

Particular preference is given to the use of 1,3-diethyl-5,6,7,8-tetrahydronaphtho[1,2-d]imidazolinone, 1-ethyl-5,6,7,8-tetrahydronaphtho[1,2-d]imidazolinone; 3-ethylnaphtho[1,2-d]oxazolinone; 3-ethylnaphtho[1,2-d]imidazolinone; 3-ethylquinolino[5,6-d]imidazolinone and 3-benzyl-(2,1,3-thiadiazolo)-[4,5-g]benzimidazolone.

Furthermore, a chemical compound in accordance with the general formula (V) (see WO 00/37422):

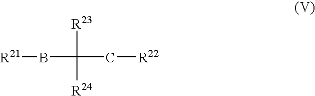

or a pharmaceutically tolerated salt, an oxide or a hydrate thereof, can be used as a modulator in accordance with the invention;

where

B and C, independently of each other, are a group of the formula —(CH₂)ₙ—, of the formula —(CH₂)ₙ—Y′— (in one of the two directions) or of the formula —(CH₂)ₙ—Y′—(CH₂)ₘ—, where, in this formula, n and m are, independently of each other, 0, 1, 2, 3 or 4, Y′ is O, S or NR″′, where R″′ is hydrogen or an alkyl group;

R²¹ and R²² are, independently of each other, alkyl, alkenyl, alkynyl, cycloalkyl, amino, trihalomethyl, in particular trifluoromethyl or trichloromethyl, nitro, cyano, phenyl or a group of the formula OR′, —SR′, —R′OR″, —R′SR″, —C(O)R′, —C(S)R′, —C(O)OR′, —C(S)OR′, —C(O)SR′, —C(S)SR′, —C(O)NR′(OR″), —C(S)NR′(OR″), —C(O)NR′(SR″), —C(S)NR′(SR″), —CH(CN)₂, —C(S)NR′R″, —C(O)NR′R″, —CH[C(O)R′]₂, —CH[C(S)R′]₂, —CH[C(O)OR′]₂, —CH[C(S)OR′]₂, —CH[C(O)SR′]₂, —CH[C(S)SR′]₂, CH₂OR′, CH₂SR′, —NR′C(O)R″, or OC(O)R′;

or is an unsaturated, partially saturated or saturated monocyclic or polycyclic group, an aralkyl group or heteroalkyl group, where the monocyclic or polycyclic groups, aralkyl groups or heteroalkyl groups can be substituted, once or more than once, by a group consisting of halogen, in particular F or Cl, trihalomethyl, in particular trifluoromethyl or trichloromethyl, alkyl, alkenyl, alkynyl, amino, nitro, cyano or amido, or a group of the formula —R′, —OR′, SR′, —R′OR″, —R′SR″, —C(O)R′, —C(S)R′, —C(O)OR′, —C(S)OR′, —C(O)SR′ or —C(S)SR′, or a phenyl group or phenoxy group, where the group, in particular the phenyl group or phenoxy group, can be substituted, once or more than once, by a group consisting of halogen, in particular F or Cl, trihalomethyl, in particular trifluoromethyl or trichloromethyl, alkyl, alkenyl, alkynyl, amino, nitro, cyano or amido, or a group of the formula —R′, —OR′, SR′, —R′OR″, —R′SR″, —C(O)R′, —C(S)R′, —C(O)OR′, —C(S)OR′, —C(O)SR′, —C(S)SR′, —NR′C(O)R″ or OC(O)R′;

where

R′ and R″ are, independently of each other, hydrogen, alkyl, alkenyl, alkynyl, cycloalkyl, alkoxy or phenyl, where appropriate substituted, in particular by F or Cl, or a group of the formula NR″′R″″, where R″′ and R″″, independently of each other, are hydrogen or alkyl;

R²³ and R²⁴ are, independently of each other, alkyl, alkenyl, alkynyl, cycloalkyl, amino, trihalomethyl, in particular trifluoromethyl or trichloromethyl, nitro, cyano, phenyl or a group of the formula OR′, —SR′, —R′OR″, —R′SR″, —C(O)R′, —C(S)R , —C(O)OR′, —C(S)OR′, —C(O)SR′, —C(S)SR′, —C(O)NR′(OR″), —C(S)NR′(OR″), —C(O)NR′(SR″), —C(S)NR′(SR″), —CH(CN)₂, —C(S)NR′R″, —C(O)NR′R″, —CH[C(O)R′]₂, —CH[C(S)R′]₂, —CH[C(O)OR′]₂, —CH[C(S)OR′]₂, —CH[C(O)SR′]₂, —CH[C(S)SR′]₂, CH₂OR′, CH₂SR′, —NR′C(O)R″, or OC(O)′;

where

R′ and R″ are, independently of each other, hydrogen, alkyl, alkenyl, alkynyl, cycloalkyl, alkoxy, phenyl or a group of the formula NR″′R″″, where R″′ and R″″ are, independently of each other, hydrogen or alkyl, where appropriate substituted, in particular by F or Cl;

or R²³ and R²⁴ together form an unsaturated, partially saturated or completely saturated monocyclic or polycyclic group or a monoheterocyclic or polyheterocyclic group, where the heteroatom is preferably N, S or O, where the monocyclic or polycyclic groups can be substituted, once or more than once, by a group consisting of halogen, in particular F or Cl, trihalomethyl, in particular trifluoromethyl or trichloromethyl, alkyl, alkenyl, alkynyl, amino, nitro, cyano or amido, or a group of the formula —R′, —OR′, SR′, —R′OR″, —R′SR″, —C(O)R′, —C(S)R′, —C(O)OR′, —C(S)OR′, —C(O)SR′, —C(S)SR′, or a phenyl group or phenoxy group, where the group, in particular the phenyl group or phenoxy group, can be substituted, once or more than once, by a group consisting of halogen, in particular F or Cl, trihalomethyl, in particular trifluoromethyl or trichloromethyl, alkyl, alkenyl, alkynyl, amino, nitro, cyano or amido, or a group of the formula —R′, —OR′, SR′, —R′OR″, —R′SR″, —C(O)R′, —C(S)R′, —C(O)OR′, —C(S)OR′, —C(O)SR′, —C(S)SR′, —NR′C(O)R″ or OC(O)R′;

where

R′ and R″ are, independently of each other, hydrogen, alkyl, alkenyl, alkynyl, cycloalkyl, alkoxy, phenyl or a group of the formula NR″′R″″, where R″′ and R″″ are, independently of each other, hydrogen or alkyl, where appropriate substituted, in particular by F or Cl.

A preferred embodiment of a modulator which can be used in accordance with the invention is a malic ester derivative in accordance with general formula (VIII)

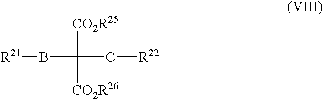

or a pharmaceutically tolerated salt, an oxide or a hydrate thereof,

where

A, B, R²¹ and R²² are defined as above and R²⁵ and R²⁶ are, independently of each other, hydrogen, alkyl, cycloalkyl, where appropriate substituted, in particular by F or Cl, or a group of the formula NR′″R″″, where R″′ and R″″ are, independently of each other, hydrogen or alkyl, where appropriate substituted, in particular by F or Cl.

In another preferred embodiment, it is possible to use a malic ester derivative as described above where R²⁵ and R²⁶ together form a heterocyclic 6-9-membered ring and produce a diester derivative.

Particular preference is given to the use, according to the invention, of the following malic acid derivatives as modulators of IK activity:

diethyl-2-(4-fluorophenyl)-2-(3-picolyl)malonate; diethyl-2-(4-nitrophenyl)-2-(2-picolyl)malonate; diethyl-2-(4-nitrophenyl)-2-(4-picolyl)malonate; diethyl-2-phenyl-2-(3-picolyl)malonate; diethyl-2-(5-chloro-2-nitro-4-(trifluoromethyl)phenyl)-2-(3-picolyl)malonate; diethyl-2-benzyl-2-(3-picolyl)-malonate; diethyl-2-(4-nitrophenyl)-2-[(benzotriazol-1-yl)methyl]malonate; diethyl-2-(2-thienyl)-2-(2-picolyl)malonate; diethyl-2-(4-(acetylamino)phenyl)-2-(2-picolyl)malonate; diethyl-2-(4-benzoylamino)phenyl)-2-(2-picolyl)malonate and 2-(4-nitrophenyl)-2-(2-picolyl)malononitrile.

Other substances in accordance with the general formula V whose use according to the invention is particularly preferred are 2-(3-phenoxyphenyl)butyronitrile; 2-(2-chlorophenyl)butyronitrile; dicyclopropane(4-chlorophenyl)carbinol; ethyl-1-(4-chlorophenyl)cyclopentane-1-carboxylate and 1-(4-chlorophenyl)-1-(3-methyl-5-oxadiazolyl)cyclopentane.

In another embodiment, the modulators which are used in accordance with the invention act as activators of IK activity.

Isatin derivatives in accordance with the following general formula (VI) (see also WO 00/33834)

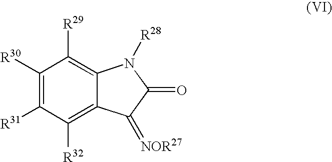

or a pharmaceutically tolerated salt, an oxide or a hydrate thereof, can be used as activators in accordance with the invention;

where

R²⁷ is hydrogen; an alkyl group; a cycloalkyl group; an acyl group; a phenyl group or benzyl group, where the group, in particular the phenyl group or benzyl group, can be substituted, once or more than once, by a group selected from halogen, in particular F or Cl, —NO₂, —CN, —CF₃, alkyl, cycloalkyl, hydroxyl and alkoxy; a group of the formula —CH₂CN; a group of the formula —CH₂CO₂R′, where R′ is hydrogen or an alkyl group, where appropriate substituted, in particular by F or Cl; a group of the formula —CH₂CONR^{IV}R^{V}, where R^{IV} and R^{V} are, independently of each other, hydrogen, alkyl, where appropriate substituted, in particular by F or Cl, phenyl group or benzyl group, where the phenyl group or benzyl group can be substituted, once or more than once, by halogen and/or alkyl, or R^{IV} and R^{V}, together with the N atom to which they are bonded, form a 4-membered to 7-membered monocyclic ring, where the heterocyclic group can be substituted, once or more than once, by a group selected from halogen, in particular F or Cl, alkyl, cycloalkyl, alkyloxy, cycloalkyloxy, phenyl or benzyl; or a group of the formula —CH₂C(═NOH)NH₂.

R²⁸ is hydrogen; an alkyl group; a cycloalkyl group; a group of the formula —CH₂CO₂R′, where R′ is hydrogen or an alkyl group; or is a phenyl group or benzyl group, where the phenyl group or benzyl group can be optionally substituted, once or more than once, by substituents selected from halogen, in particular by F or Cl, —NO₂, —CN, CF₃, alkyl, cycloalkyl, hydroxyl and alkoxy; and

R²⁹, R³⁰, R³¹ and R³² are, independently of each other, hydrogen; halogen, in particular by F or Cl; —NO₂; —CN; CF₃; alkyl; an alkoxy group; a phenyl group or benzyl group, where the phenyl or benzyl groups can be substituted, once or more than once, by substituents selected from halogen, in particular by F or Cl, —NO₂, —CN, CF₃, alkyl, cycloalkyl, hydroxyl and alkoxy; or a group of the formula —SO₂NR″R″′, where R″ and R″′ are, independently of each other, hydrogen or an alkyl group, where appropriate substituted, in particular by F or Cl;

or R³¹ and R³² are defined as above, and R²⁹ and R³⁰ together form an additional 4-membered to 7-membered fused ring, where the fused ring can be aromatic, partially saturated or saturated and the fused ring can be substituted, once or more than once, by substituents selected from halogen, in particular by F or Cl, —NO₂, —CN, CF₃ and a group of the formula —SO₂NR″R″′, where R″ and R″′ are, independently of each other, hydrogen or an alkyl group, where appropriate substituted, in particular by F or Cl.

In a preferred embodiment, R²⁷ in formula (VI) is hydrogen; a C₁₋₆ alkyl group; a phenyl group; a benzyl group; a group of the formula —CH₂CO₂R′, where R′ is hydrogen or a C₁₋₆ alkyl group, where appropriate substituted, in particular by F or Cl; a group of the formula —CH₂NH—Z, where Z is a phenyl or benzyl group, where these phenyl or benzyl groups can be substituted, once or more than once, by halogen, in particular by F or Cl; or a group of the formula CH₂CO—Y″, where Y″ is a heterocyclic 6-membered monocyclic group containing at least one nitrogen atom as heteroatom, and which can be substituted, once or more than once, by a C₁₋₆ alkyl group or a phenyl group. Y″ is preferably a piperidinyl group or a piperazinyl group.

In another preferred embodiment, R²⁸ in the formula (VI) is hydrogen, C₁₋₆ alkyl group, phenyl, benzyl or a group of the formula —CH₂COOH.

In another preferred embodiment, R²⁹, R³⁰, R³¹ and R³² are, independently of each other, hydrogen, F, Br, Cl, NO₂, CN, CF₃ or C₁₋₆ alkyl, where appropriate substituted, in particular by F or Cl.

Particularly preferred modulators which can be used in accordance with the invention are: 5,7-dinitro-1-methyl-1H-indole-2,3-dione-3-(O-methyloxime); 5-bromo-7-nitro-1H-indole-2,3-dione-3-oxime; 5-bromoisatin-3-oxime; 5,6-dichloro-1-methylisatin-3-oxime; 4,5-dichloroisatin-3-oxime; 4,5-dichloro-1-methylisatin-3-oxime; O-methyl-4,5-dichloro-1-methylisatin-3-oxime; benzoisatin-3-oxime; 6,7-dichloroisatin-3-oxime; O-methyl-6,7-dichloroisatin-3-oxime; O-methyl-6,7-dichloro-1-methylisatin-3-oxime; 6,7-dichloro-1-methylisatin-3-oxime; O-t-butyl-6,7-dichloroisatin-3-oxime; 0-((4-phenylpiperazin-1-yl)carbonylmethyl)-6,7-dichloroisatin-3-oxime; 6-fluoro-7-methoxyisatin-3-oxime; 0-(4-chlorobenzylamino)methyl-6,7-dichloroisatin-3-oxime; 6,7-difluoroisatin-3-oxime; 6,7-dimethylisatin-3-oxime; 5,6-dichloroisatin-3-oxime; O-carboxymethyl-5-bromoisatin-3-oxime; 0-(ethoxycarbonylmethyl)-5-bromoisatin-3-oxime; 0-(carboxymethyl)-6,7-dichloroisatin-3-oxime; 0-(carboxymethyl)-6,7-dichloroisatin-3-oxime; 6-chloro-7-methylisatin-3-oxime; 6-fluoro-7-methylisatin-3-oxime.

Another modulator which can be used in accordance with the invention is zoxazolamine, which acts as activators of hIK1 (Syme et al., 2000, Am. J. Physiol. 278: C570-C581).

Within the meaning of the invention, halogen is a fluorine, chlorine, bromine or iodine atom. Particularly preferred halogens are F or Cl.

Within the meaning of the invention, an alkyl group is a monovalent, saturated, unbranched or branched hydrocarbon chain. The hydrocarbon chain preferably contains from 1 to 12 carbon atoms (C₁₋₁₂-alkyl), in particular from 1 to 6 carbon atoms (C₁₋₆-alkyl; lower alkyl), containing pentyl, isopentyl, neopentyl, tertiary pentyl, hexyl and isohexyl. In an even more preferred embodiment, alkyl is a C₁₋₄ alkyl group, containing butyl, isobutyl, secondary butyl and tertiary butyl. In a particularly preferred embodiment, alkyl is a C₁₋₃-alkyl group which can, in particular, be methyl, ethyl, propyl or isopropyl.

Within the meaning of the present invention, a cycloalkyl group is a cyclic alkyl group which preferably contains from three to seven carbon atoms (C₃₋₇-cycloalkyl), including cyclopropyl, cyclobutyl, cyclopentyl and cyclohexyl.

Within the meaning of the invention, an alkoxy group is an ‘alkyl-O—’ group, with alkyl being defined as above.

Within the meaning of the present invention, an amino group can be a primary (—NH₂), secondary (—NH—R) or tertiary amino group (—N—R′R″), where R′ and R″ are, independently of each other, alkyl as defined above.

Within the meaning of the present invention, an acyl group is a carboxyl group or an alkylcarbonyl group, with alkyl being defined as above. Examples of preferred acyl groups are carboxyl, acetyl and propionyl.

Within the meaning of the present invention, an alkenyl group is a carbon chain containing one or more double bonds, including dienes, trienes and polyenes. In a preferred embodiment, the alkenyl group of the present invention contains between two and six carbon atoms (C₂₋₆-alkenyl). Particularly preferred alkenyl groups are ethenyl, 1, 2 or 2,3-propenyl; or 1,2-, 2,3- or 3,4-butenyl.

Within the meaning of the present invention, an alkynyl group denotes a carbon chain containing one or more triple bonds, including diynes, triynes and polyynes. In a preferred embodiment, the alkynyl group of the present invention contains between two and six carbon atoms (C₂₋₆-alkynyl). Particularly preferred alkynyl groups are ethynyl, 1, 2 or 2,3-propynyl; or 1,2-, 2,3- or 3,4-butynyl.

Within the meaning of the present invention, an amido group is a substituent of the formula R′—CO—NH— or R′—CO—N(alkyl)-, where R′ is hydrogen or an alkyl group defined as above. Examples of preferred amido groups are formamido, acetamido and propionamido.

Within the meaning of this invention, a monocyclic or polycyclic aryl group is a monocyclic or polycyclic aromatic hydrocarbon group. Examples of preferred aryl groups are phenyl, naphthyl and anthracenyl.

Within the meaning of this invention, an unsaturated monocyclic or polycyclic group is a monocyclic or polycyclic alkyl group, i.e. a monocyclic or polycyclic aromatic hydrocarbon chain. An example of a preferred, partially saturated monocyclic group is cyclopenta-2,4-dien-1-ylidene.

Within the meaning of the invention, an aralkyl group is an aryl group defined as above, with the aryl group being linked to an alkyl group defined as above. An example of a preferred aralkyl group is benzyl.

Within the meaning of the invention, a monocyclic or heterocyclic group is a monocyclic or polycyclic compound which contains one or more heteroatoms in the ring structure. Preferred heteroatoms contain nitrogen (N), oxygen (O) and sulfur (S). One or more ring structures can be aromatic (i.e. heteroaryl), saturated or partially saturated. Preferred monocyclic groups contain 5-membered and 6-membered heterocyclic groups. Examples of preferred monocyclic heterocyclic groups contain furan-2-yl; furan-3-yl; 2-, 4- or 5-imidazolyl, 3-, 4-, or 5-isoxazolyl, 1-, 2-, 3-pyridinyl and 1- or 2-thienyl. Examples of preferred saturated or partially saturated monocyclic heterocyclic groups contain 1,3,5,6,2-dioxadiazinyl, piperazinyl, piperidinyl, 1,2-, 1,3- or 1,4-pyranyl and pyrrolidinyl. Examples of preferred aromatic heterocyclic groups contain acridinyl, carbazolyl, indazolyl, quinolinyl or benzofuranyl.

Within the meaning of the invention, a heteroalkyl group denotes a monoheterocyclic or polyheterocyclic group as defined above, with the heterocyclic group being linked to an alkyl group defined as above.

Particularly preferred modulators which can be used in accordance with the invention are hIK4 activators 1-EBIO (1-ethyl-2-benzimidazolinone), DCEBIO (5,6-dichloro-1-ethyl-1,3-dihydro-2H-benzimidazol-2-one) and also the benzoxazolones chlorzoxazone and zoxazolamine, which act as activators of hIK1 (Syme et al., 2000, Am.

J. Physiol. 278: C570-C581). In addition, WO 00/33834 discloses isatin derivatives as activators of hIK1. Examples are 5-bromoisatin-3-oxime and 5,6-dichloro-1-methylisatin-3-oxime.

Particularly preferred inhibitors of hIK4 are charybdotoxin and clotrimazole (Khanna et al., 1999, J. Biol. Chem. 274: 14838-14849). WO 99/25347 discloses additional substances which act as inhibitors of hIK1. Examples are the imidazole derivatives miconazole, econazole, butoconazole, oxiconazole, sulconazole and thioconazole, the triazole derivatives fluconazole, terconazole and itraconazole, and the nitroimidazole derivatives metronidazole, tinidazole, nimorazole, ornidazole and benznidazole. In addition, the document discloses derivatives of 1,4-dihydropyridine-3,5-dicarboxylic acid, for example ethylmethyl 1,4-dihydro-2,6-dimethyl-4-(2-nitrophenyl)pyridine-3,5-dicarboxylate, and also clotrimazole derivatives, for example 2-chlorophenyl-4-hydroxyphenylphenylmethane.

The modulators which are used in accordance with the invention can be employed for diagnosing and/or preventing and/or treating diseases which are connected with disturbed keratinocyte activity, in particular wound healing disturbances, particularly preferably psoriasis. Preferred diseases which are connected with disturbed keratinocyte activity are contact dermatitis, atopic eczema, vitiligo, hyperkeratoses, actinic keratoses, hypertrophic scars, keloids, lentigo, ephelides and geriatric skin. Particularly preferred diseases are wounds, for example normally healing wounds and poorly healing wounds, in particular ulcera, and also psoriasis.

The present invention also relates to a process for producing a pharmaceutical for treating diseases which are connected with disturbed keratinocyte activity, in particular wound healing disturbances, particularly preferably psoriasis, in which process at least one nucleic acid which can be used in accordance with the invention, at least one polypeptide which can be used in accordance with the invention, at least one antibody which can be used in accordance with the invention and/or at least one modulator which can be used in accordance with the invention is combined with suitable additives and auxiliary substances.

In the case of psoriasis, or preference is to be given to using a modulator of the activity of the ion channel, for example an activator of the activity of the ion channel or an inhibitor of the activity of the ion channel in accordance with the present invention. Examples of modulators are DCEBIO, zoxazolamine, chlorzoxazone, 1-EBIO and a specific antibody. In addition, preference is to be given, in the case of psoriasis, to a nucleic acid which leads to a downregulation of the quantity of IK channel, for example an antisense oligonucleotide, an siRNA or a ribozyme in accordance with the present invention.

In the case of wound healing, in particular of ulcera, preference is to be given to increasing the quantity of IK channel, for example by means of carrying out a gene-therapy treatment using a nucleic acid which encodes a polypeptide which can be used in accordance with the invention.

The present invention furthermore relates to a pharmaceutical, which has been produced by this process, for treating diseases which are connected with disturbed keratinocyte activity, in particular wound healing disturbances and/or psoriasis, particularly preferably psoriasis, which pharmaceutical comprises at least one polypeptide, as depicted in SEQ ID No. 3 or SEQ ID No. 4, or a one nucleic acid encoding this sequence, or or at least one antibody which can be used in accordance with the invention, or at least one modulator which can be used in accordance with the invention, where appropriate together with suitable additives and auxiliary substances.

The invention furthermore relates to the use of this pharmaceutical for treating diseases which are connected with disturbed keratinocyte activity.

Diseases which are connected with disturbed keratinocyte activity can be treated in a conventional manner, for example using bandages, plasters, compresses or gels which contain the pharmaceuticals according to the invention. Thus, it is possible to administer the pharmaceuticals, which comprise suitable additives or auxiliary substances, such as physiological sodium chloride solution, demineralized water, stabilizers, proteinase inhibitors, gel formulations, such as white vaseline, low-viscosity paraffin and/or yellow wax, etc., topically and locally in order to immediately and directly exert an influence on the disease. Furthermore, the pharmaceuticals according to the invention can, where appropriate, be administered in the form of liposome complexes or gold particle complexes, likewise topically and locally in the region of the diseased skin surface. In addition, the treatment can be effected using a transdermal therapeutic system (TTS) which enables the pharmaceuticals according to the invention to be released in a time-controlled manner. TTSs are disclosed, for example, in EP 0 944 398 A1, EP 0 916 336 A1, EP 0 889 923 A1 and EP 0 892 493 A1. However, the treatment with the pharmaceuticals according to the invention can also be effected using oral dosage forms, such as tablets or capsules, by way of the mucous membranes, for example of the nose or of the oral cavity, or in the form of dispositories which are implanted under the skin.

A preferred embodiment of a pharmaceutical is used for preventing and/or treating psoriasis, with the intention being to decrease the activity of the keratinocytes. An embodiment which is particularly preferred in this connection consists in using suitable modulators and/or antibodies to inhibit the (electrophysiological) activity of the hIK1 channel according to the invention.

A particularly preferred embodiment consists in administering a medicament which comprises a modulator which can be used in accordance with the invention [lacuna] can be effected in the form of the active form of the modulator. Preference is given to administering a composition which comprises the active form of the modulator, optionally in the form of a pharmaceutically acceptable salt, together with one or more suitable additives and auxiliary substances, such as adjuvants, carrier materials and buffer solutions and diluents, such as physiological sodium chloride solution, demineralized water, stabilizers, proteinase inhibitors, gel formulations, such as white vaseline, low-viscosity paraffin and/or yellow wax, etc., which are particularly suitable for topical application in order to immediately and directly exert an influence on the wound healing.

A preferred embodiment consists in using an antibody, or an antibody fragment, which is directed against the hIK1 channel polypeptide, or a functional variant thereof, as a pharmaceutical in accordance with the present invention.

Another particularly preferred embodiment consists in decreasing the expression of the nucleic acids which encode the hIK1 polypeptide according to the invention.

A preferred embodiment of a nucleic acid as a pharmaceutical in accordance with the present invention is represented by antisense nucleotides (see above) and ribozymes (WO 99/15703) and also what are termed small interfering RNAs (siRNAs). The latter are double-stranded RNA molecules which downregulate gene expression in a sequence-specific and post-transcriptional manner (Elbashir et al., 2001, Nature 411: 494-498).

Another preferred embodiment of a pharmaceutical is used for preventing and/or treating wound healing disturbances by, for example, administering a polypeptide according to the invention or (a) nucleic acid(s) which encode(s) the polypeptide.

A pharmaceutical which comprises the described nucleic acid in naked form or in the form of one of the above-described vectors which are effective in gene therapy, or in a form in which it is complexed with liposomes or gold particles, is especially suitable for use in performing gene therapy in humans. The pharmaceutical excipient is, for example, a physiological buffer solution, preferably having a pH of approx. 6.0-8.0, preferably of approx. 6.8-7.8. In particular of approx. 7.4 and/or an osmolarity of approx. 200-400 milliosmol/liter, preferably of approx. 290-310 milliosmol/liter. In addition, the pharmaceutical excipient can comprise suitable stabilizers such as nuclease inhibitors, preferably sequestrants such as EDTA, and/or other auxiliary substances with which the skilled person is familiar.

The described nucleic acid, where appropriate in the form of the viral vectors which are described in more detail above, or as liposome complexes or a gold particle complex, is customarily administered topically and locally in the region of the wound.

A preferred embodiment of a pharmaceutical in accordance with the present invention consists in administering the polypeptide itself, together with suitable additives or auxiliary substances, such as physiological sodium chloride solution, demineralized water, stabilizers, proteinase inhibitors, gel formulations, such as white vaseline, low-viscosity paraffin and/or yellow wax, etc., in order to immediately and directly exert an influence on the wound healing.

Another preferred embodiment of a pharmaceutical in accordance with the present invention consists in using a suitable carrier material, for example what are termed microcarriers, which are composed of biocompatible materials, such as dextran matrix (see U.S. Pat. No. 598,888), to transplant autologous and allogenic cells which expresses the polypeptide according to the invention.

The present invention furthermore relates to a process for producing a diagnostic agent for diagnosing diseases which are connected with disturbed keratinocyte activity, in particular wound healing disturbances and/or psoriasis, which process is characterized in that use is made of at least one nucleic acid, as depicted in SEQ ID Nos. 1 and 2, of at least one polypeptide, as depicted in SEQ ID Nos. 3 and 4, or of at least one modulator in accordance with the present invention, where appropriate together with suitable additives and auxiliary substances.

The present invention also relates to a diagnostic agent for diagnosing diseases which are connected with disturbed keratinocyte activity, in particular wound healing disturbances and/or psoriasis, which agent comprises at least one nucleic acid, as depicted in SEQ ID Nos. 1 and 2, at least one polypeptide, as depicted in SEQ ID Nos. 3 and 4, or at least one modulator in accordance with the invention, where appropriate together with suitable additives and auxiliary substances.

For example, it is possible, in accordance with the present invention, to use one of the above-described nucleic acids to prepare a diagnostic agent on the basis of the polymerase chain reaction (Examples 3, 4, 5; PCR diagnostics, e.g. in accordance with EP 0 200 362) or of an RNase protection assay, as described in more detail in Example 2. These tests are based on the specific hybridization of the above-described nucleic acids with the complementary counterstrand, usually the corresponding mRNA. In this connection, the above-described nucleic acid can also be modified as described, for example, in EP 0 063 879. Preferably, well known methods employing suitable reagents are used to label, for example radioactively using α-P³²-dCTP or nonradioactively using biotin or digoxigenin, an above-described DNA fragment, which is then incubated with isolated RNA which has preferably previously been bound to suitable membranes composed, for example, of cellulose or nylon. When the quantity of RNA being analyzed is the same for each tissue sample, it is consequently possible to determine the quantity of mRNA which has been specifically bound by the probe and to compare this with the quantity of mRNA derived from healthy tissue. Alternatively, in situ hybridization can also be used to determine the quantity of mRNA in tissue sections (see, for example, Werner et al., 1992, Proc. Natl. Acad. Sci. USA 89: 6896-900).

The diagnostic agent according to the invention can consequently also be used to specifically measure a tissue sample in vitro for the strength of the expression of the corresponding gene in order to be able to diagnose a possible disease which is connected with disturbed keratinocyte activity with certainty (Examples 2, 3, 4 and 5). Such a method is particularly suitable for the early prognosis of disturbances. This makes it possible to use therapy preventatively and to analyze predispositions. Thus, expression of the gene mIK1 is already increased in the unwounded state in the intact skin of old mice, which exhibited wound healing disturbances after wounding, as compared with the intact skin of control mice and, in particular, with the intact skin of young mice. The strength with which the mIK1 gene is expressed therefore makes it possible to still predict wound healing disturbance in tissue which is intact (Example 3, Table 2).

Another diagnostic agent according to the invention comprises the polypeptide which can be used in accordance with the invention or the immunogenic parts thereof which are described in more detail above. For example, the polypeptide, or the parts thereof, which are preferably bound to a solid phase, for example composed of nitrocellulose or nylon, can, for example, be brought into contact, in vitro, with the body fluid, for example wound secretion, to be investigated in order, in this way, to be able to react, for example, with autoimmune antibody. The antibody/peptide complex can then be detected, for example using labeled anti-human IgG antibodies or anti-human IgM antibodies. The label is, for example, an enzyme, such as peroxidase, which catalyzes a color reaction. The color reaction can then be used to readily and rapidly detect the presence and quantity of autoimmune antibodies present.

Another diagnostic agent comprises the antibodies according to the invention themselves. These antibodies can be used, for example, to readily and rapidly investigate a tissue sample to determine whether the polypeptide in question is present in elevated quantity in order, thereby, to obtain an indication of the possible presence of a disease which is connected with disturbed keratinocyte activity. In this case, the antibodies according to the invention are, for example, labeled with an enzyme, as already described above. The specific antibody/peptide complex can thereby be detected readily, and just as rapidly, by way of an enzymic color reaction.

Another diagnostic agent according to the invention comprises a probe, preferably a DNA probe, and/or primers. This opens up a further possibility of using a suitable probe to obtain the nucleic acids which can be used in accordance with the invention, for example by isolating them from a suitable gene library, for example from a wound-specific gene library (see, for example, J. Sambrook et al., 1989, Molecular Cloning, A Laboratory Manual, 2nd edition, Cold Spring Harbor Laboratory, Cold Spring Harbor, N.Y., chapter 8, pages 8.1 to 8.81, chapter 9, pages 9.47 to 9.58 and chapter 10, pages 10.1 to 10.67).

Examples of suitable probes are DNA or RNA fragments having a length of approx. 100-1000 nucleotides, preferably having a length of approx. 200-500 nucleotides, in particular having a length of approx. 100-400 nucleotides, and whose sequences can be deduced from the polypeptide sequences, or functional variants thereof, as depicted in SEQ ID No. 3 or SEQ ID No. 4 of the sequence listing and/or with the aid of the cDNA sequences, or variants thereof, as depicted in SEQ ID No. 1 or SEQ ID No. 2 [lacuna] specified database entries (see also Example 2).

Alternatively, it is possible to use the deduced nucleic acid sequences to synthesize oligonucleotides which are suitable for being used as primers for a polymerase chain reaction. These primers can be used to amplify and isolate the nucleic acid which can be used in accordance with the invention, or parts thereof, from cDNA, for example skin-specific cDNA (Examples 2, 3, 4 and 5). Examples of suitable primers are DNA fragments having a length of approx. 10-100 nucleotides, preferably having a length of from approx. 10 to 50 nucleotides, in particular having a length of 10-30 nucleotides, whose sequence can be deduced from the polypeptides as depicted in SEQ ID No. 3 to SEQ ID No. 4 and/or with the aid of the cDNA sequences as depicted in SEQ ID No. 1 or SEQ ID No. 2 (Examples 3, 4 and 5).

The invention also relates to a process for producing a test for finding pharmacologically active substances which are connected with diseases involving disturbed keratinocyte activity, in particular wound healing disturbances and/or psoriasis, which process is characterized in that at least one nucleic acid, as depicted in SEQ ID Nos. 1 and 2, at least one polypeptide, as depicted in SEQ ID Nos. 3 and 4, at least one antibody or at least one modulator in accordance with the present invention is/are used, where appropriate together with suitable additives and auxiliary substances, for producing the test.

Within the meaning of the present invention, the term ‘pharmacologically active substances’ is to be understood as meaning all those molecules, compounds and/or compositions and substance mixtures which are able to interact, under suitable conditions, where appropriate together with suitable additives and auxiliary substances, with the above-described nucleic acids, polypeptides or antibodies. While possible pharmacologically active substances are simple chemical organic or inorganic molecules or compounds, they can also contain peptides, proteins or complexes thereof. Because of their interaction, the pharmacologically active substances can exert an influence, in vivo or in vitro, on the function(s) of the nucleic acids, polypeptides or antibodies or else only bind to the above-described nucleic acids, polypeptides or antibodies or enter into other interactions, in a covalent or noncovalent manner, with them. In particular, pharmacologically active substances are substances which are able to modulate the channel activity as described above.

In addition, the invention contains a test for identifying substances which are pharmacologically active in connection with diseases which are connected with disturbed keratinocyte activity, which test includes at least one nucleic acid, at least one polypeptide or at least one modulator according to the present invention, where appropriate together with suitable additives and auxiliary substances.

A suitable system can be produced, for example, by stably transforming epidermal or dermal cells, in particular keratinocytes, with expression vectors which contain selectable marker genes and the above-described nucleic acids. In this method, the expression of the above-described nucleic acids in the cells is altered such that it corresponds to the pathologically disturbed expression in vivo. It is also possible to employ antisense oligonucleotides, which contain the nucleic acids which can be used in accordance with the invention, for this purpose. In the case of these systems, therefore, it is particularly advantageous to be acquainted with the expression behavior of the genes in disturbed physiological processes as described in this application. The pathological behavior of the cells in vitro can frequently be imitated in this way and it is possible to search for substances which restore the normal behavior of the cells and which possess therapeutic potentential.

HaCaT cells, which are generally available, and the expression vector pCMV4 (Anderson et al., 1989, J. Biol. Chem. 264: 8222-9) are, for example, suitable for these test systems. In this connection, the nucleic acid which can be used in accordance with the invention can be integrated into the expression vectors either in the sense orientation or in the antisense orientation such that the functional concentration in the cells of the mRNA of the corresponding genes is either increased or decreased as a result of hybridizing with the antisense RNA. After the transformation and selection of stable transformands the cells in culture generally exhibit a proliferation behavior, migration behavior and/or differentiation behavior which is/are altered as compared with control cells. This behavior in vitro frequently correlates with the function of the corresponding genes in regenerative processes in the organism (Yu et al., 1997, Arch. Dermatol. Res. 289: 352-9; Mils et al., 1997, Oncogene 14: 15555-61; Charvat et al., 1998, Exp. Dermatol 7: 184-90; Mythily et al., 1999, J. Gen. Virol. 80: 1707-13; Werner, 1998, Cytokine Growth Factor Rev. 9: 153-65) and can be detected using simple tests which are easy to implement such that, based on this, it is possible to construct test systems for identifying pharmacologically active substances. Thus, the proliferative behavior of cells can be established very rapidly by, for example, incorporating labeled nucleotides into the DNA of the cells (see, for example, de Fries and Mitsuhashi, 1995, J. Clin. Lab. Anal. 9: 89-95; Perros and Weightman, 1991, Cell Prolif. 24: 517-23; Savino and Dardenne, 1975, J. Immunol. Methods 85: 221-6), by staining the cells with specific dyes (Schulz et al., 1994, J. Immunol. Methods 167: 1-13) or by using immunological methods (Frahm et al., 1998, J. Immunol. Methods 211: 43-50). Migration can be readily detected using the migration index test (Charvat et al., see above) and comparable test systems (Benestad et al., 1987, Cell Tissue Kinet. 20: 109-19, Junger et al., 1993, J. Immunol. Methods 160: 73-9). Examples of suitable differentiation markers are keratin 6, 10 and 14 and also loricrin and involucrin (Rosenthal et al., 1992, J, Invest, Dermatol, 98: 343-50), whose expression can readily be detected, for example, using antibodies which are available generally.

Another suitable test system is based on identifying interactions using what is termed the two hybrid system (Fields and Sternglanz, 1994, Trends in Genetics, 10, 286-292; Colas and Brent, 1998 TIBTECH, 16, 355-363). In this test, cells are transformed with expression vectors which express fusion proteins composed of the polypeptide which can be used in accordance with the invention and a DNA-binding domain from a transcription factor such as Gal4 or LexA. In addition, the transformed cells contain a reporter gene whose promoter contains sites for binding the corresponding DNA-binding domain. By means of transforming another expression vector which expresses a second fusion protein composed of a known or unknown polypeptide and an activation domain, for example from Gal4 or herpesvirus VP16, the expression of the reporter gene can be greatly increased if the second fusion protein interacts with the polypeptide which can be used in accordance with the invention. This increase in expression can be used for identifying novel pharmacologically active substances by, for example, preparing a cDNA library from regenerating tissue for the purpose of constructing the second fusion protein. In addition, this test system can be used for screening substances which inhibit an interaction between the polypeptide which can be used in accordance with the invention and a pharmacologically active substance which is already known. These substances decrease the expression of the reporter gene in cells which are expressing the fusion proteins composed of the polypeptide which can be used in accordance with the invention and the pharmacologically active substances (Vidal and Endoh, 1999, Trends in Biotechnology, 17: 374-81). In this way, it is possible to rapidly identify novel active compounds which can be used for treating diseases which are connected with disturbed keratinocyte activity.

Pharmacologically active substances of the polypeptides which can be used in accordance with the invention can also be nucleic acids which are isolated using selection methods such as SELEX (see Jayasena, 1999, Clin. Chem. 45: 1628-50; Klug and Famulok, 1994, M. Mol. Biol. Rep. 20: 97-107; Toole et al., 1996, U.S. Pat. No. 5,589,281). In the SELEX method, those molecules (aptamers) which bind to a polypeptide with high affinity are typically isolated, by means of repeated amplification and selection, from a large pool of different, single-stranded RNA molecules. Aptamers can also be synthesized and selected in their mirror-image form, for example as a L-ribonucleotide (Nolte et al., 1996, Nat. Biotechnol. 14: 1116-9; Klussmann et al., 1996, Nat. Biotechnol. 14: 1112-5). Forms which have been isolated in this way enjoy the advantage that they are not broken down by naturally occurring ribonucleases and therefore possess greater stability.

The invention also relates to a process for preparing an array, which is fixed on a support material, for performing analysis in connection with diseases which are connected with disturbed keratinocyte activity, in which process at least one nucleic acid, at least one polypeptide or at least one antibody in accordance with the invention is used for the preparation.

Methods for preparing such arrays by means of spotting, printing or solid phase chemistry in combination with photolabile protecting groups are disclosed, for example, in WO 89/109077, WO 90/15070, WO 95/35505 and U.S. Pat. No. 5,744,305.

The invention furthermore relates to an array, which is fixed on a support material, for performing analysis in connection with diseases which are connected with disturbed keratinocyte activity, in particular wound healing disturbances and/or psoriasis, which array is characterized in that it contains at least one nucleic acid and/or at least one polypeptide and/or at least one antibody in accordance with the present invention. Arrays according to the invention can be used for performing analysis in connection with diseases which are connected with disturbed keratinocyte activity, in particular wound healing disturbances and/or psoriasis.

The invention furthermore relates to a process for preparing a DNA chip and/or protein chip for performing analysis in connection with diseases which are connected with disturbed keratinocyte activity, in particular wound healing disturbances and/or psoriasis, which process is characterized in that at least one nucleic acid, as depicted in SEQ ID Nos. 1 and 2, at least one polypeptide, as depicted in SEQ ID Nos. 3 and 4, or at least one antibody, as described above, is used for the preparation.

Methods for preparing such DNA chips and/or protein chips by means of spotting, printing or solid phase chemistry in combination with photolabile protecting groups are disclosed, for example, in WO 89/109077, WO 90/15070, WO 95/35505 and U.S. Pat. No. 5,744,305.

The invention furthermore relates to a DNA chip and/or protein chip for performing analysis in connection with diseases which are connected with disturbed keratinocyte activity, in particular wound healing disturbances and/or psoriasis, which DNA chip and/or protein chip contain(s) at least one nucleic acid, as depicted in SEQ ID Nos. 1 and 2, and/or at least one polypeptide, as depicted in SEQ ID Nos. 3 and 4, and/or or at least one antibody in accordance with the present invention. DNA chips are disclosed, for example, in U.S. Pat. No. 5,837,832.

The present invention also relates to a pharmaceutical for indication and therapy, which pharmaceutical comprises a nucleic acid which can be used in accordance with the invention, a polypeptide which can be used in accordance with the invention or a modulator which can be used in accordance with the invention and, where appropriate, suitable additives or auxiliary substances, and to a process for producing such a pharmaceutical for treating diseases which are connected with disturbed keratinocyte activity, in particular wound healing disturbances and/or psoriasis, in which process a nucleic acid which can be used in accordance with the invention, as depicted in SEQ ID Nos. 1 and 2, a polypeptide which can be used in accordance with the invention, as depicted in SEQ ID Nos. 3 and 4, or a modulator which can be used in accordance with the invention, is formulated with a pharmaceutically acceptable excipient.

The wording of all the claims is hereby incorporated into the description by reference.

The following tables and figures, and the following examples, are solely intended to describe the invention in more detail without restricting it.

### TABLES AND FIGURES

Table 1 lists the hIK1 polypeptides and nucleic acids which can be used in accordance with the invention and their database accession numbers.

Table 2 shows differential regulation of mIK1 expression, as determined by means of TaqMan analysis, in association with different wound healing states in the mouse as an example of diseases which are connected with disturbed keratinocyte activity.

Table 3 shows the kinetics of the differential regulation of mIK1 expression, as determined by means of TaqMan analysis, in association with wound healing as an example of a disease which is connected with disturbed keratinocyte activity.

Table 4 shows differential regulation of mIK1 expression, as determined by means of TaqMan analysis, in two different wound healing states in humans (normal wound healing and ulcer) as examples of diseases which are connected with disturbed keratinocyte activity. Table 5 shows the expression profile of hIK1, as determined by means of TaqMan analysis, in different human organs.

FIG. 1 shows the change in membrane potential (Vm) in mV, as determined by means of patch-clamp analysis, as the electrophysiological response of HaCaT keratinocytes to (A) different concentrations of ATP, (B) 10 μM bradykinin or 100 μM histamine (C)′ caged IP₃′ before and after UV-induced flash photolysis.

FIG. 2 shows the influence, as determined by means of patch-clamp analysis, of different modulators of hIK1 on the ATP-induced electrophysiological response as a change in the membrane potential (Vm) in mV. (A) addition of charybdotoxin at various concentrations prior to the addition of ATP (B) addition of clotrimazole (1 μM) prior to the addition of ATP (C) addition of charybdotoxin (100 nM) after the addition of ATP (D) addition of 1-EBIO (1 mM) prior to the addition of ATP.

FIG. 3 shows the immunostaining of just confluent (confluent), 2 days postconfluent (2d postconfluent), 4 days postconfluent (4d postconfluent) and 6 days postconfluent (6d postconfluent) HaCaT cells with an anti-K10 antibody.

FIG. 4 shows the quantity of hIK1, as determined by means of an RNase protection assay, in proliferating (lane ‘1’), just confluent (lane ‘2’) and 4 days postconfluent (lane ‘3’) HaCaT keratinocytes as an autoradiograph. 1000 cpm of the hybridization probe are loaded in the ‘probe’ lane. The ‘tRNA’ lane contains the negative control.

FIG. 5 shows the influence of various modulators of hIK1 on the proliferation of HaCaT keratinocytes (n=3). As a control, use was made of cells where only the respective solvent was added to the medium. Clotrimazole (10 μM) and 1-EBIO (1 mM) inhibited proliferation completely while the addition of charybdotoxin (200 nM) retarded proliferation.

FIG. 6 shows the quantities, as detected by means of immunoblotting, of the protein markers of keratinocyte differentiation, i.e. involucrin, keratin 1 (K1) and keratin 10 (K10) under the influence of 1000 μM 1-EBIO (lanes 1 and 2), 100 μM 1-EBIO (lanes 3 and 4), 1 μM 1-EBIO (lanes 5 and 6), 1000 μM zoxazolamine (lanes 7 and 8), 100 μM zoxazolamine (lanes 9 and 10), 1 μM zoxazolamine (lanes 11 and 12), and in the control (lane ‘C’; 1% DMSO/DMEM), in HaCaT keratinocytes.

FIG. 7 shows the influence of different concentrations of the hIK1 activator 1-EBIO on the proliferation of HaCaT keratinocytes without addition of serum. The incorporation of BrDU was determined, as a measure of the proliferation, by measuring the absorption at 450 nm (A[450 nm]). Greater proliferation is reflected in higher absorption. Cells which had grown under the influence of serum (‘KGM-10% FCS’ lane) were used as the proliferation control. Another hIK1 activator, i.e. zoxazolamine (‘1000 μM Zox’ lane) was used for comparing with the effect of 1-EBIO. The negative controls, without activator or only with the solvent DMSO, are labeled “KGM” and ‘KGM DMSO’, respectively.

FIG. 8 shows the influence of different concentrations of the hIK1 activator 1-EBIO on the serum-induced proliferation of HaCaT keratinocytes (‘DMEM-10% FCS’ lane). The incorporation of BrDU was determined, as a measure of the proliferation, by measuring the absorption at 450 nm (A[450 nm]). Greater proliferation is reflected in higher absorption. DMSO in DMEM/10% FCS without activator was used as the negative control for the solvent (‘DMSO’ lane). The hIK1 activator zoxazolamine, in DMEM/10% FCS, was used as the positive control (‘1000 μM Zox’ lane).

The polypeptide sequences of mIK1 and hIK1, and their encoding nucleic acids, are as depicted in SEQ ID No. 1 to SEQ ID No. 4.

The oligonucleotides and polynucleotides which were used for the examples are as depicted in SEQ ID No. 5 to SEQ ID No. 13.

### EXAMPLES

### Example 1

### The Intermediate-conductance Calcium-dependent Potassium Channel hIK1 Mediates a Long-lasting, 1-EBIO-Modulated Hyperpolarization of Keratinocytes Following Stimulation with ATP, Bradykinin or Histamine

In order to identify an ion channel which is substantially involved in the transduction of wound healing-specific signals, HaCaT cells were investigated electrophysiologically. The HaCaT keratinocytes were propagated in DEM containing 10% FCS (Gibco BRL) to which antibiotics (penicillin/streptomycin, 100 U/ml) had been added. The final concentration of Ca²⁺ was 1.8 mM. The cells were incubated until a confluent cell culture had developed; the culture medium was then replaced with standard bath solution (SBS; 130 mM NaCl, 3 mM KCl; 2 mM CaCl₂; 2 mM MgCl₂; 25 mM HEPES/NaHEPES; 10 mM D-glucose; pH 7.4), and the dishes were examined in an inverse microscope (Axiovert, Zeiss). The patch pipettes were prepared from small borosilicate tubes on a horizontal pulling device (DMZ, Zeitz) using the two-step pulling method. The pipettes were then filled with a solution containing 135 mM potassium gluconate, 10 mM KCl, 1.6 mM Na₂HPO₄, 0.73 mM CaCl₂, 1.03 mM MgCl₂, 1 mM EGTA, 14 mM HEPES/NaHEPES and 100 mg of nystatin/ml; pH 7.2 (Ca²⁺ activity in the solution=10⁻⁷ M). The resistance of these patch pipettes was 5-8 GΩ in the bath. After a seal in the GΩ range had been produced (typically 1.5-2 GΩ) the amplifier was switched to current clamp mode. The membrane potentials reached stable values within 3-5 min. The electrophysiological signals were recorded, amplified and digitalized using an Axopatch 200 amplifier in combination with a TL-1 Labmaster Interface and AXOTAPE software (Axon Instruments). The statistical analysis (one-way ANOVA with Bonferri post-test comparison) was carried out using Graphpad prism 2.0.

In experiments in which the intracellular Ca²⁺ concentration was increased by flash photolysis of ‘caged IP₃’ (Calbiochem), the patch-clamp recording was carried out in the whole-cell configuration, with a pipette solution containing 120 mM potassium gluconate, 20 mM KCl, 1 mM MgCl₂, 10 mM HEPES/NaHEPES, 2 mM Na₂ATP, 0.3 mM NaGTP (pH 7.2) and 10-100 mM ‘caged IP₃’ being used. AFter 5-7 min of whole-cell recording, IP3 was released by means of a UV light flash which was generated using a xenon arc lamp (75 W; excitation filter: 330±40 nm) which was connected to the epifluorescence port of the microscope. The UV light beam was focused on the keratinocytes by the 40-times phase contrast objective. The duration of the flash (1 s) was set using a Uniblitz shutter (Vincent Assoc.).

In experiments in which the entire solution in which the cells were incubated was exchanged, a conical plexiglas holder possessing several influx lines and one efflux line was placed in the culture dish in order to reduce the volume of the recording chamber down to 1 ml. The inflow of liquid was gravity-dependent and was controlled by means of magnetic valves. Outflow was effected by applying a negative pressure using a vacuum pump. Substances were added, while observing visually, directly into the keratinocytes under investigation using a remotely controlled, magnet-controlled Y tube system (diameter of the efflux tube: 100 μm).

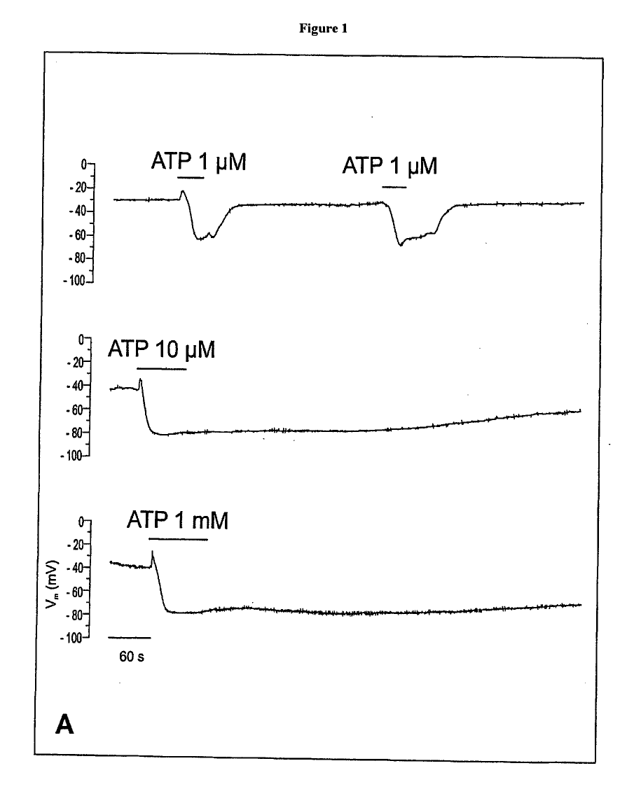

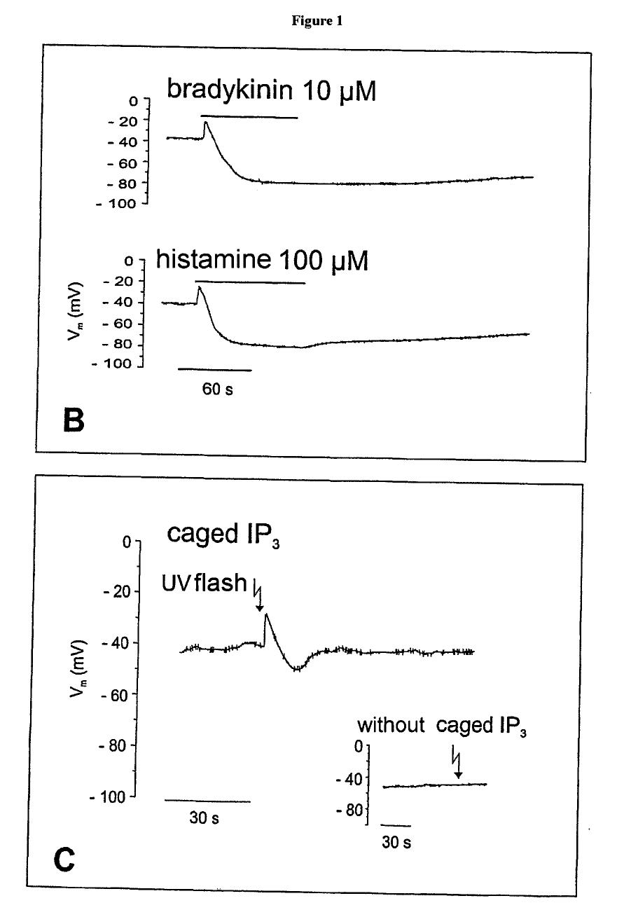

The electrophysiological recordings were carried out, using perforated patches, on HaCaT cells which had grown into a confluent monolayer. In the current-clamp mode, the average membrane potential of the keratinocytes was −42±1 mV (n=76) under controlled conditions. Perfusing the HaCaT cells with ATP (1-1000 μM) induced a biphasic change in the membrane potential, with this change consisting of a short, transient depolarization followed by a marked and long-lasting hyperpolarization (FIG. 1A). At ATP concentrations of ≧10 μM, the strong hyperpolarization lasted for at least 5 min after addition of the ATP (5 min), with some keratinocytes even remaining hyperpolarized during the whole of the observation period (up to 20 min). This observation demonstrates that changes in the concentration of ATP bring about long-lasting changes in the membrane potential and consequently in the activity of the keratinocytes.

The hyperpolarization after the addition of 1 μM ATP represented a process which could be reversed rapidly by washing out the solution (n=9). When 10 μM ATP were added, a hyperpolarization of 30±2 mV (n=28) was observed. A response which was practically identical quantitatively and kinetically was measured when bradykinin (10 μM, n=3) or histamine (100 μM, n=4) was added to the keratinocytes, indicating that the substances ATP, histamine and bradykinin regulate the same effector system (FIG. 1B).

Since it is known that all three extracellular mediators are able to stimulate phosphoinositide metabolism in keratinocytes (Talwar et al., 1989, J. Invest. Dermatol. 93: 241-245; Pillai et al., 1992, J. Clin. Invest. 90: 42-51; Rosenbach et al., 1993, Arch. Dermatol. Res. 285: 393-396), an investigation was carried out to determine whether the formation of IP₃ and the subsequent increase in the cytosolic concentration of Ca²⁺ are part of the signal transduction pathway. To do this, whole-cell recordings were carried out using keratinocytes which had been loaded with ‘caged-IP₃’ (10-100 μM). In all the cells which were investigated (n=7), it was observed that the sudden conversion of ‘caged-IP₃’ into IP₃ by means of flash photolysis led to a rapid, transient depolarization followed by a hyperpolarization (FIG. 1C). The IP₃-induced biphasic voltage change tallied very precisely with that which was observed after adding 1 μM ATP. The striking similarity between the electrophysiological responses which were brought about by the three agonists, i.e. ATP, histamine and bradykinin, and IP₃ suggest that the biphasic change in membrane voltage was mediated by the mobilization of Ca²⁺ from IP₃-sensitive stores.

Since the three extracellular mediators, as well as the IP₃-mediated signal pathway, might also be able to activate other signal cascades, an investigation was carried out to determine whether stimulation of adenylyl cyclase (AC) or protein kinase C (PKC) imitate or influence the ATP-mediated electrophysiological response.

Pretreating the HaCaT cells with the AC activator forskolin (10 μM) for 3-10 min did not have any effect either on the resting potential or the changes in voltage which are observed after adding ATP (n=5). While stimulating PKC with the phorbol ester PMA (60 ng/μl) caused a slow depolarization to −32 ±2 mV, it had no influence on the effect of ATP (n=4). While inhibiting PKC with calphostin C (50 nM) in turn led to a weak hyperpolarization to −58±3 mV, it had likewise no effect on the ATP-mediated change in voltage.

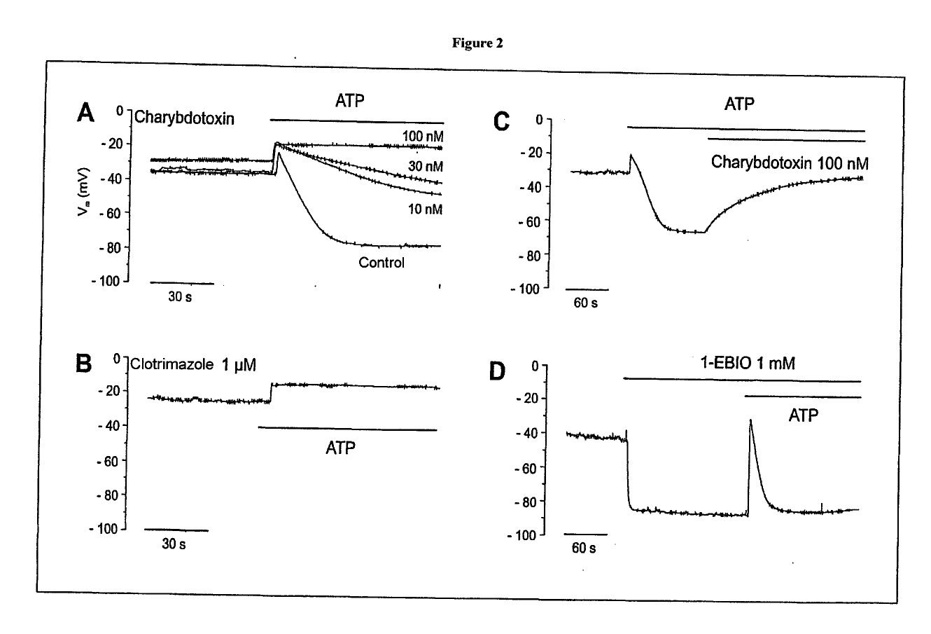

Calcium-dependent potassium channels are possible candidates which may be responsible for this marked and long-lasting change, which has been demonstrated for the first time, in the membrane potential following stimulation with the signal molecules ATP, histamine and bradykinin. In order to identify the ion channel which was responsible for the hyperpolarization, an investigation was carried out into the influence of various K⁺ channel blockers on the ATP-mediated electrophysiological effect. Charybdotoxin (ChTx; Alomone Labs) caused a dose-dependent inhibition of the ATP-mediated hyperpolarization. As FIG. 2A shows, the addition of ChTx (10-100 nM, n=15) retarded repolarization of the rapid depolarization and prevented hyperpolarization. When ChTx was used at a concentration of 100 nM (n=4), it completely prevented repolarization and the membrane potential remained on a depolarized plateau as long as ATP was added. The same effect was observed with 1 μM clotrimazole (n=3; Figure 2 B). If ChTx (100 nM) was added after the ATP-mediated hyperpolarization had been measured, a complete reversal of the hyperpolarization was observed even though ATP was still present (n=2; FIG. 2C). None of the other K⁺ channel blockers which were tested (TEA, Ba²⁺ and verapamil) led to a comparable depolarization on a plateau during treatment with ATP. Verapamil (10-100 μM), which inhibits K⁺ flows in terminally differentiating keratinocytes (Mauro et al., 1997, J. Invest. Derm., 108: 864-870), did not have any influence on the hyperpolarizing effect of ATP (hyperpolarization to −73±1 mV, n=3). K⁺ channels which are inhibited by clotrimazole and charybdotoxin are voltage-independent calcium-activated potassium channels. In order to clarify the identity still further, an investigation was carried out to determine whether 1-EBIO, which is a specific activator of the intermediate-conductance calcium-activated potassium channel hIK1 (Syme et al., 2000, Am. J. Physiol., 278: C570-581), is able to imitate ATP-mediated hyperpolarization. As FIG. 2D shows, 1-EBIO (1 mM; tocris) induced a rapid and powerful hyperpolarization to −83±3 mV (n=3). If ATP (10 μM) was added in the presence of 1-EBIO, the typical transient depolarization was observed, with this depolarization being much stronger, however, than under controlled conditions (cf. FIG. 1A), something which can be explained by the increased flow of chloride ions and cations due to the increase in voltage across the membrane. It was furthermore observed that a derivative of 1-EBIO, i.e. DCEBIO (5,6-dichloro-1-ethyl-1,3-dihydro-2H-benzimidazol-2-one) can elicit a depolarization of the HaCaT cells which is 300-1000 times stronger than that elicited by 1-EBIO. This result shows that DCEBIO can be used at much lower concentrations in order to achieve the same electrophysiological effect as that achieved with 1-EBIO (approx. 300-1000 times less concentrated) and is consequently a particularly preferred modulator of the hIK1 channel.

It was consequently possible, for the first time, to detect hIK1 in keratinocytes and to demonstrate its role in keratinocyte activity. This demonstrates that the polypeptide hIK1 and its encoding nucleic acids, and also modulators of the channel, are suitable for being used in diseases which are connected with disturbed keratinocyte activity, in particular in wound healing disturbances and/or psoriasis.

### Example 2

### Detecting the Expression of hIK1 in HaCaT Cells and Differential Regulation of hsk4 (═IK1) in Differentiating Keratinocytes

In order to investigate whether the expression, as well as the activity, of hIK1 is regulated in keratinocytes, a check was carried out to determine whether the activity of the keratinocytes correlates to the expression of hIK1.

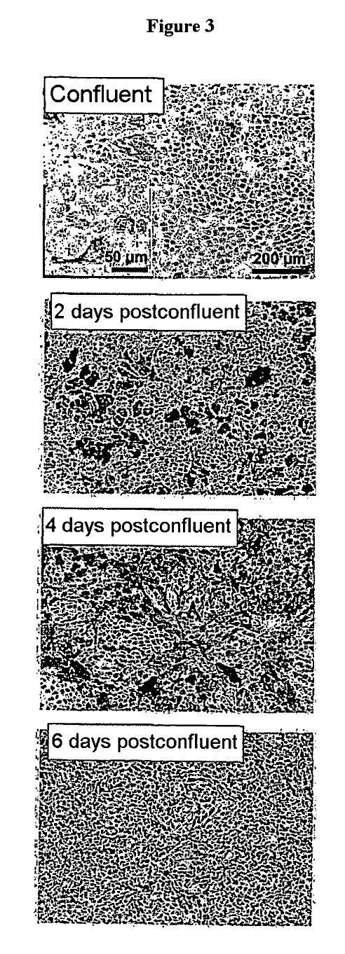

For this, an analysis was carried out of proliferating subconfluent cells, of cells which had just reached confluence and of resting cells (4 days after confluence). The activity of these keratinocytes was determined using the differentiation-specific expression of keratin 10. To do this, the cells were washed in ice-cold PBS and fixed with acetone/methanol (1:1) at −20° C. for 20 min. Endogenous peroxidases were blocked with 1% H₂O₂ at room temperature. After the nonspecific binding sites had been saturated with 3% BSA in PBS, the cells were incubated overnight at 4° C. in a 1:500 dilution of mouse anti-K10 antibody (DAKO) in 3% BSA in PBS and then washed three times with PBS and once with 3% BSA in PBS. After a 2 h incubation at room temperature with a peroxidase-coupled anti-mouse antibody, the cells were washed three times with PBS and once with ddH₂O. The cells were then stained using the peroxidase substrate kit and 3-amino-9-ethylcarbazole as the chromogenic substrate (Vector Laboratories). Whereas the proliferating cells, and the cells which had just reached confluence, did not exhibit any staining, it was found that the majority of the postconfluent cells were stained (FIG. 3). The expression of the differentiation-specific keratin 10 gene showed that the majority of the resting cells consisted of partially differentiated cells (Fuchs, 1988, Trends Genet. 4: 277-281; Ryle et al., 1989, Differentiation, 40: 42-54).

Standard methods were used to isolate RNA from proliferating, confluent and postconfluent cells which had been grown in parallel (Chomczynski and Sacchi, 1987, Anal. Biochem. 62: 156-159), and an RNAse protection assay was carried out for detecting hIK1 (Werner et al., 1992, Proc. Natl. Acad. Sci. USA 89: 6896-6900).

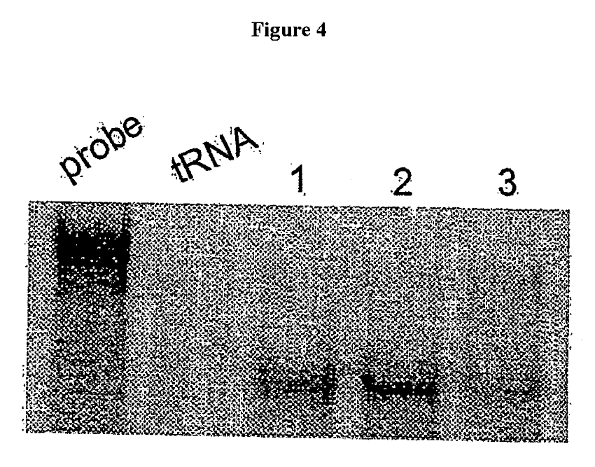

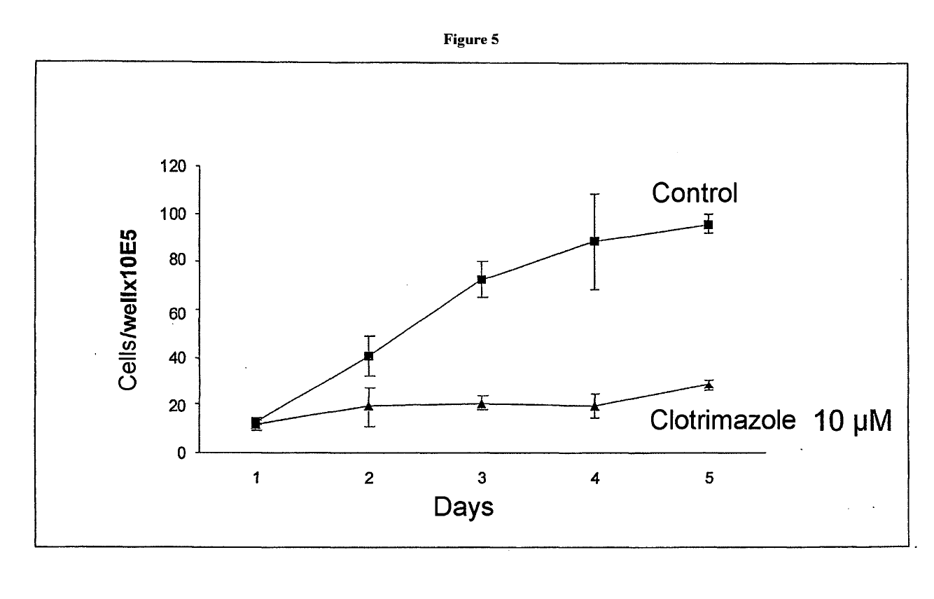

In brief: a PCR-amplified gene fragment was cloned into the transcription vector pBluescript KSII (+) (Stratagene) and the plasmid was linearized. An antisense transcript was then prepared in vitro using T3 or T7 polymerase and ³²P-UTP (800 Ci/mol). In each case, 20 μg of RNA were hybridized overnight at 42° C. with 100 000 cpm of labeled antisense transcript. 20 μg of tRNA were used as the negative control. The hybrids were then digested at 30° C. for 1 h with RNAse and RNAse T1. The protected fragments were fractionated on 5% acrylamide/8 M urea gels and analyzed by autoradiography. A riboprobe (SEQ ID No. 5) which was complementary to nucleotides 740-1004 of the human hIK1 gene was used for detecting hIK1. The result of the experiment is shown in FIG. 4. It was observed that hIK1 is strongly expressed in proliferating (lane ‘1’) and just confluent cells (lane ‘2’) whereas the quantity was significantly reduced in postconfluent, partially differentiated cells (lane ‘3’). No signal was observed in the case of the negative control (‘tRNA’ lane). 1000 cpm of the hybridization probe were loaded, as a control for the radioactive label and as a size standard, in the ‘probe’ lane. The results, which were repeated three times using RNAs from independent experiments, therefore show that the onset of differentiation in HaCaT keratinocytes is accompanied by a reduction in hIK1 mRNA and there is consequently a connection between keratinocyte activity and hIK1 expression.

### Example 3

### Differential Expression of mIK1 in Mouse Wounds

The skin is a highly complex system whose cell composition is subjected, for example during a disease, to spatial and chronological changes. Moreover, the activities of the cells change, for example as a result of interaction with other cells. Processes in the skin can therefore only be inadequately imitated by simple cell systems. In order to confirm that IK1 plays an important role in regulating cell activities in physiological processes in the skin as well as in cell culture, the expression of murine mIK1 (SEQ ID No. 2) was monitored in biopsies of punch wounds taking wound healing, and various wound healing states, as the example. The investigation was effected by means of TaqMan analysis in an Applied Biosystems GeneAmp 5700. Normally healing day 1 wounds and intact skin were obtained from isotonic sodium chloride solution-treated 10-week-old BALB/c mice by cutting with scissors. In order to obtain tissue from mice possessing poorly healing wounds, BALB/c mice were treated with dexamethasone (0.5 mg of dexamethasone in isotonic salt solution per kg of body weight i.p. twice daily for 5 days) prior to the wounding. The control employed was intact skin from mice which had been treated with isotonic sodium chloride solution as described above. In order to obtain tissue from young mice and old mice, use was made of untreated day 1 wounds and intact skin samples from 4-week-old and 12-month-old BALB/c mice. Wound tissue and intact skin from mice with diabetes (db/db mouse) were obtained by isolating untreated day 1 wounds and intact skin from 10-week-old C57B/Ks db/db/Ola mice by cutting with scissors. C57B/Ks wild-type mice were used as control animals in this experiment. Intact skin and untreated day 1 wounds were also obtained from these latter animals.

The RNA was isolated by using a disperser to homogenize the biopsies in RNAclean buffer (AGS, Heidelberg) to which 1/100th part by volume of 2-mercaptoethanol had been added. The RNA was then extracted by being phenolized twice with water-saturated acidic phenol in the presence of 1-bromo-3-chloropropane. An isopropanol precipitation and an ethanol precipitation were then carried out and the RNA was washed with 75% ethanol. After that, the RNA was subjected to digestion with DNase I. For this, 20 μg of RNA (to 50 μl with DEPC-treated water) were incubated, at 37° C. for 20 min, with 5.7 μl of transcription buffer (Roche), 1 μl of RNase inhibitor (Roche; 40 U/μl) and 1 μl of DNase I (Roche; 10 U/μl). 1 μl of DNase I was then added once again and the mixture was incubated at 37° C. for a further 20 min. After that, the RNA was phenolized, precipitated with ethanol and washed. All the steps enumerated above were carried out with DEPC (diethylpyrocarbonate)-treated solutions or liquids provided the latter did not contain any reactive amino groups. cDNA was then prepared from the extracted RNA. This took place in the presence of 1× TaqMan RT buffer (PE Applied Biosystems), 5.5 mM MgCl₂ (PE Applied Biosystems), in each case 500 μM dNTPs (PE Applied Biosystems), 2.5 μM random hexamers (PE Applied Biosystems), 1.25 U of MultiScribe reverse transcriptase/μl (50 U/μl, PE Applied Biosystems), 0.4 U of RNase inhibitor/μl (20 U/μl, PE Applied Biosystems), 20 μl of RNA (50 ng/μl) and DEPC-treated water (to make up to a volume of 100 μl). After the RNA had been added, and after thorough mixing, the mixture was aliquoted into 2×0.2 ml tubes (in each case 50 μl) and the reverse transcription was carried out in a temperature cycler (10 min at 25° C.; 30 min at 48° C. and 5 min at 95° C.).

The cDNA was subsequently quantified by means of quantitative PCR using the ‘SYBR-Green PCR Master Mix’ (PE Applied Biosystems) with a triple determination (in each case using mIK1 primers and GAPDH primers) being carried out for determining the quantity of the mIK1 cDNA. The stock solution for each triplet contained, in a total volume of 57 μl, 37.5 μl of 2×‘SYBR Master Mix’, 0.75 μl of AmpErase UNG (1 U/μl) and 18.75 μl of DEPC-treated water. For each triplicate determination, 1.5 μl each of forward primer and backward primer (mIK1 primer 1 (SEQ ID No. 6) and mIK1 primer 2 (SEQ ID No. 7)) were added, in a previously optimized concentration ratio, to 57 μl of stock solution. In each case 60 μl of the stock solution/primer mixture were mixed with 15 μl of cDNA solution (2 ng/μl) and the whole was aliquoted into 3 wells. In parallel with this, a stock solution containing primers for determining GAPDH (SEQ ID No. 12 and SEQ ID No. 13) was prepared as a reference, mixed with a further 15 μl of the same cDNA solution and aliquoted into 3 wells. In addition, different cDNA solutions were prepared as a dilution series in order to construct a standard curve for the GAPDH PCR (4 ng/μl; 2 ng/μl; 1 ng/μl; 0.5 ng/μl and 0.25 ng/μl). In each case, 15 μl volumes of these cDNA solutions were mixed with 60 μl of stock solution/primer mixture for determining GAPDH and aliquoted into 3 wells. A standard curve for the mIK1 PCR was constructed in the same way; the same dilutions were used as those for the GAPDH standard curve. A PCR mixture without cDNA served as the control. In each case, 15 μl of DEPC water were added to in each case 60 μl of mIK1 and GAPDH stock solution/primer mixture and the total mixture was in each case aliquoted into 3 wells. The assay mixtures were amplified in a GeneAmp 5700 (2 min at 50° C.; 10 min at 95° C., followed by 3 cycles of 15 s at 96° C. and 2 min at 60° C.; after that 37 cycles of 15 s at 95° C. and 1 min at 60° C.).

The analysis was effected by determining the relative abundance of mIK1 in relation to the GAPDH reference. To do this, a standard curve was first of all constructed, with the C_{T} values for the dilution series being plotted against the logarithm of the quantity of cDNA in the PCR assay mixture (in ng of transcribed RNA) and the slope (s) of the straight lines being determined. The efficiency (E) of the PCR is then given as follows: E=10^{-1/s}−1. The relative abundance (X) of the mIK1 cDNA species under investigation (Y) in relation to GAPDH is then: X=(1+E_{GAPDH})^{C}_{T}^{(GAPDH)}/(1+E_{Y})^{C}_{T}^{(Y)}. The numerical values were then standardized by making the quantity of cDNA from the intact skin of 10-week-old BALB/c control animals, or the intact skin of the C57B/Ks control animals, equal to 1. The relative changes in the expression of mIK1 in various wound healing states are compiled in Table 2. It turns out that it was possible to observe an increase in mIK1 expression, as compared with the intact skin, in all the wound healing states in the mouse. A striking feature was that, while a marked upregulation, by a factor of 3 and a factor of 4, respectively, was measured in the control animal wound and the wound which healed well in young mice, only a weak, and nonsignificant, increase in expression was observed in the wounds in old mice. A less marked increase in expression as compared with control animals (factor, 2.2) was also observed in poorly healing, dexamethasone-treated wounds. This underlines the fact that, in murine wounds, wound healing is accompanied by a marked increase in mIK1 expression and that disturbed keratinocyte activity, as can occur to an increased extent in the wounds of older or dexamethasone-treated animals, can be characterized by a decrease in mIK1 expression. In general, therefore, modulating, preferably increasing, the expression and/or activity of IK1 can be advantageous for treating or preventing diseases which are connected with disturbed keratinocyte activity.

In order to determine suitable time points for diagnosing, preventing and/or treating a disease connected with disturbed keratinocyte activity, the expression of mIK1 was determined in murine biopsies taken at different time points in wound healing. In this experiment, wound biopsies, and a biopsy of the intact skin of 10-week-old control mice, were obtained by cutting with scissors, as described above, at the times of 1 h, 7 h, 15 h, 24 h, 3 days, 5 days, 7 days and 14 days after wounding. The RNA was isolated from the tissue and the relative quantity of mIK1 in the biopsies was determined. In this case, it turned out that it was already possible to observe a marked upregulation of mIK1 expression at 7 h after wounding (Table 3). The expression of mIK1 remained increased, as compared with intact skin, up to the end of the period of observation (day 14 after wounding), indicating that regulation of mIK1 expression over the entire course of the wound healing is essential for the wound healing to proceed optimally. This underlines the particular suitability of IK1 channels for diagnosing, preventing and/or treating diseases which are connected with disturbed keratinocyte activity since a diagnosis can be carried out, or the prevention and/or treatment can be begun, at any time point.

### Example 4

### Differential Expression of hIK1 in Human Wounds

Mice have proved to be a suitable model system for investigating wound healing in humans. Despite this, the intention was to check whether it was also possible to detect in humans a differential expression of the nucleic acids which can be used in accordance with the invention, which was observed in mice when wound healing was taken as the example. To do this, 4 mm or 6 mm punches were used to take skin samples from the untreated intact skin, from day 1 wounds or from day 5 wounds of healthy volunteers. The biopsies taken from in each case 14 volunteers were pooled in the case of each group (intact skin, day 1 wound and day 5 wound). In addition, punch biopsies of the intact skin, and of the bottom of the wound and the edge of the wound, were taken simultaneously in patients suffering from chronic venous ulcers (ulcera cruris venosum). The biopsies from in each case 6 volunteers were pooled in the case of each group (intact skin, wound edge and wound bottom). The biopsies were comminuted using a ball mill and, as described in Example 3, RNA was then isolated from the comminuted biopsies, as described in Example 3, after which the RNA was digested with DNase I and then transcribed into cDNA. Wound healing-relevant cDNAs were also quantified as described in Example 3. The results of the experiments are compiled in Table 4. The amplification primers (hGAPDH primer 1: CATGGGTGTGAACCATGAGAAG (SEQ ID No. 10); hGAPDH primer 2: CTAAGCAGTTGGTGGTGCAGG (SEQ ID No. 11), hIK1 primer 1: GCGCTCTCAATCAAGTCCG (SEQ ID No. 8), hIK1 primer 2: GTGTTCATGTAAAGCTTGGCCA (SEQ ID No. 9)) for the analysis of hIK1 were selected on the basis of the known sequences of human GAPDH (GenBank: M17851) and human hIK1 (SEQ ID No. 1). For the quantification, cDNA from 10 ng of reverse-transcribed total RNA were amplified, per assay, in a total volume of 25 μl. The PCR was carried out in accordance with the manufacturer's instructions (PE Applied Biosystems, SYBR Green PCR and RT-PCRReagents Protocol, 1998). The C_{T} values were analyzed and the frequency of hIK1 mRNA relative to GAPDH mRNA was calculated from them. The results of the experiment are shown in Table 4. It was found that the quantity of hIK1 mRNA in human day 1 wounds was only slightly increased as compared with intact skin while a decrease in expression of hIK1 was measured on day 5 after wounding, something which can be the result, for example, of the increased differentiation of the keratinocytes (see Example 2). On the other hand, it is not possible to observe any decrease in expression, as compared with intact skin, in the bottom of the ulcer patient wound in which keratinocyte activity is markedly disturbed. This demonstrates that regulation of keratinocyte activity is essential for avoiding diseases which are connected with disturbed keratinocyte activity and that hIK1 is therefore a suitable therapeutic target. In general, therefore, modulating, preferably increasing, the expression and/or activity of IK1 can be advantageously used for treating or preventing diseases which are connected to disturbed keratinocyte activity. Preferably, the expression of IK1 should be increased in wounds, in particular ulcers, of the skin.

### Example 5

### Localizing hIK1 in Different Organs

hIK1 has already been shown to be expressed in different human tissues (Ishii et al. 1997; Proc. Natl. Acad. Sci. USA 94: 11651-11656; Joiner et al. 1997; Proc. Natl. Acad. Sci. USA 94: 11013-11018; WO 99/03882). It is noticeable that expression is restricted to organs that are not electrically excitable, such as placenta and prostate, while no expression has been detected in the brain or in the heart. This is reflected in the school of thought that expression of hIK1 in humans is restricted to cells of the blood and of the vascular system (WO 00/34248). Because of the selective expression in non-excitable organs, modulators of this channel are regarded as being suitable medicaments for a large number of diseases in a variety of administration forms since the danger of cardiovascular complications is assumed to be low. Despite the large number of publications which deal with the localization of hIK1 in tissues or cells, expression in the skin has not previously been demonstrated either in humans or in another mammal.

In order to investigate whether, and in what quantity, hIK1 is expressed in the skin relative to other organs, use was made of organ-specific total RNA pools which were obtained from adult human donors (BioChain Institute, Inc.): total kidney RNA was isolated from 5 donors, while total lung RNA was isolated from 5 donors, total fat tissue RNA from 4 donors, total liver RNA from 5 donors, total brain RNA from 5 donors, total heart RNA from 5 donors, total spleen RNA from 2 donors, total skeletal muscle RNA from 5 donors, and total small intestine RNA from 5 donors, with these total RNAs then being digested with DNAse I and pooled separately according to organ. A TaqMan analysis was then carried out, as described in Example 3, using the total RNAs from these organs and total RNA from skin, which was obtained as in Example 4, and the frequency of hIK1 mRNA relative to GAPDH mRNA was determined. The relative quantities of hIK1 mRNA in the organs are shown in Table 5. It was surprisingly possible to demonstrate, for the first time, that hIK1 is expressed in the skin. In addition, it was observed that hIK1 is expressed in the skin to a degree which is comparable with that in the lung and spleen, for example. A comparatively strong expression of hIK1 had been detected in the latter two organs (WO 99/03882). In addition, it was surprisingly possible to demonstrate that hIK1 is expressed in brain, heart and skeletal muscle. This result, which is contradictory to the prior art, is probably due to the fact that the sensitivity of the TaqMan analysis is higher than that of the Northern blot analysis which was used in the prior art. This experiment therefore highlights two aspects of the present invention. On the one hand, the expression hIK1 is a prerequisite for being able to use the hIK1 gene, the protein, and modulators of its activity, for diagnosing, preventing and/or treating diseases which are connected with disturbed keratinocyte activity. On the other hand, the surprising demonstration of the expression of hIK1 in excitable organs shows that, contrary to the prevailing opinion, it is probable that cardiovascular complications will occur in association with oral administration, for example. By contrast, the prevention and/or treatment of diseases which are connected with disturbed keratinocyte activity, in particular wound healing disturbances and/or psoriasis, are preferably to be treated topically, since this form of administration ensures direct and specific contact with the keratinocytes. The use of hIK1, its encoding nucleic acids and/or modulators of its activity, is therefore particularly advantageous when preventing and/or treating these diseases.

### Example 6

### Both Activation and Inhibition of hIK1 Activity Exert an Influence on Keratinocyte Activity

The intention now is to demonstrate that modulators of the IK channel are able to regulate keratinocyte activity.

The influence of modulators on differentiation was investigated taking the hIK1 activator 1-EBIO (Syme et al., 2000, Am. J. Physiol., 278: C570-C581) as an example. For this, HaCaT cells were grown for 2 days in medium. 1 mM 1-EBIO (tocris) was then added to the medium containing the proliferating, subconfluent cells and the whole was incubated for 3 days. The differentiation of the keratinocytes was then investigated by immunostaining with a K10 antibody, as shown in Example 2. It was found that the majority of the control cells, to which no 1-EBIO had been added, were partially differentiated, with this being demonstrated by immunostaining using the anti-K10 antibody as in Example 2. The proportion of stained cells was signficantly reduced in the case of cells which were incubated in the presence of 1-EBIO.

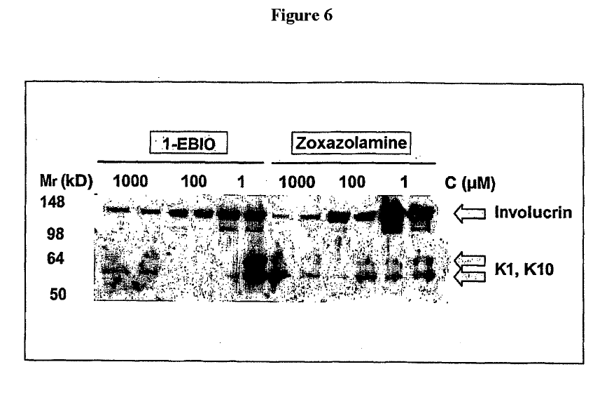

These observations were confirmed by means of Western blot analyses. For this, 5×10⁵ HaCaT cells per well were grown in medium in a 6-well plate. On the following day, the medium was replaced with medium which contained different concentrations of 1-EBIO or 2-amino-5-chlorobenzoxazole (zoxazolamine (Aldrich)). 1% DMSO in medium was used as the solvent control (lane ‘C’). The cells were incubated for 4 days, with the medium being replaced daily. After 4 days, the protein concentration in the cell lysates was determined (BCA Assay, Pierce). Immunoblotting was then used to compare equal quantities of protein from the different lysates with the keratinocyte differentiation markers involucrin (1:500, Sigma) and keratin 1 and 10 (1:250, Progen) (FIG. 6). It can be clearly seen that the two hIK1 activators, i.e. 1-EBIO and zoxazolamine, inhibit differentiation, and consequently the keratinocyte activity, of HaCaT cells in a dose-dependent manner.

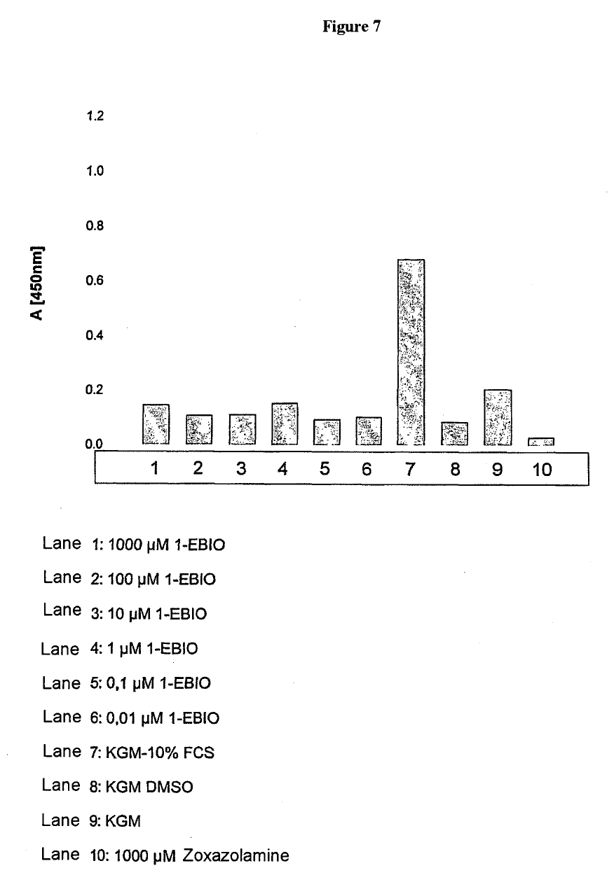

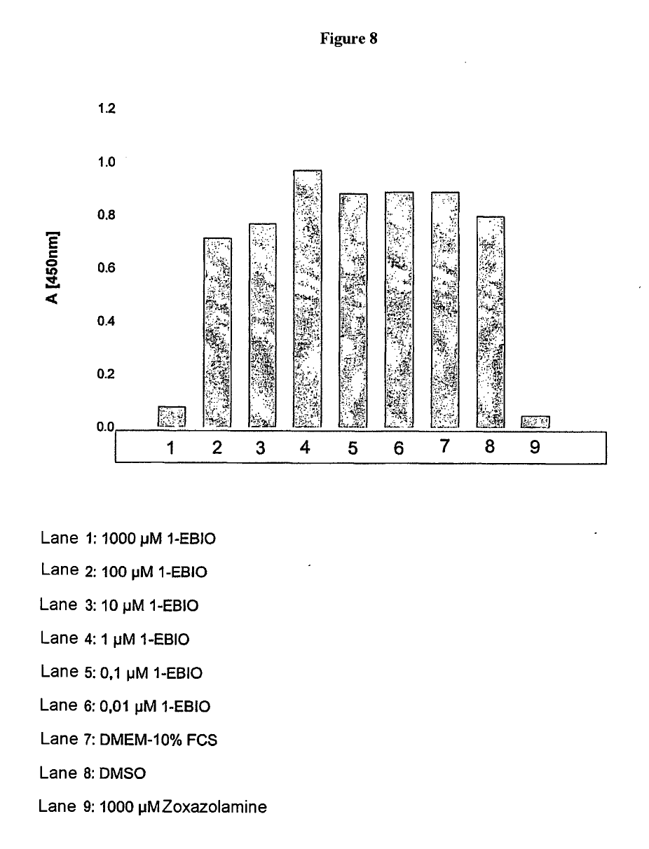

The influence of activators of IK1 on proliferation was investigated using 1-EBIO and zolxazolamine (see above). Firstly, the influence of different concentrations of 1-EBIO on the growth rate of HaCaT cells was measured in the presence and in the absence of serum. For this purpose, 1×10⁴ HaCaT cells per well were cultured in keratinocyte growth medium (KGM) for 5 days in a 96-well plate, in order to synchronize the cell cycle, with this being followed by a two-day incubation in the presence of different concentrations of 1-EBIO in KGM (see FIGS. 7 and 8) in the absence (FIG. 7) and in the presence (FIG. 8) of serum. KGM/10% FCS was used as a positive control for proliferation while the IK1 activator zoxazolamine was used at a concentration of 1 mM as a comparison for the 1-EBIO effect. 1% DMSO in KGM served as the solvent control. Cell proliferation was measured by the incorporation of bromodideoxyuridine (BrDU) while cell division was measured in accordance with the manufacturer's instructions (‘The Cell Proliferation ELISA, BrDU, Roche). The incorporation of BrDU was quantified by determining the absorption at 450 nm. In FIGS. 7 and 8, the absorption (A[450 nm]) is in each case plotted on the Y axis as a measure of the proliferation rate. Greater absorption reflects greater proliferation. Comparison of the two IK1 activators 1-EBIO and zoxazolamine between the serum-treated cells and the untreated cells clearly shows that, in the first place, the two activators are not on their own able to stimulate cell proliferation (FIG. 7). In addition to this, both activators are able to inhibit serum-induced proliferation and are consequently able to modulate keratinocyte activity (FIG. 8). In this connection, it should be mentioned that the morphology of the cells remains unaltered, i.e. the cells ‘persist’ in a resting state and there is no sign of apoptosis. FIG. 8 also shows that the inhibition brought about by the hIK1 activator 1-EBIO is dose-dependent and shows its strongest effect at a concentration of 1 mM.

In order to investigate the effect of the hIK1 channel inhibitors clotrimazole and charybdotoxin, either 10 μM clotrimazole, 200 nM charybdotoxin (ChTx) or 1 mM 1-EBIO were/was added to cells which were growing subconfluently and the number of cells per well was determined daily.

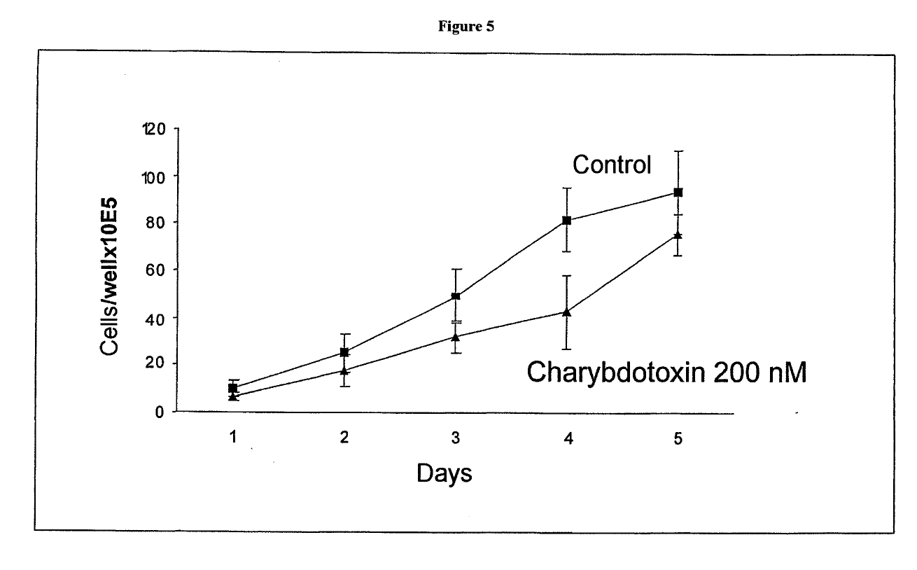

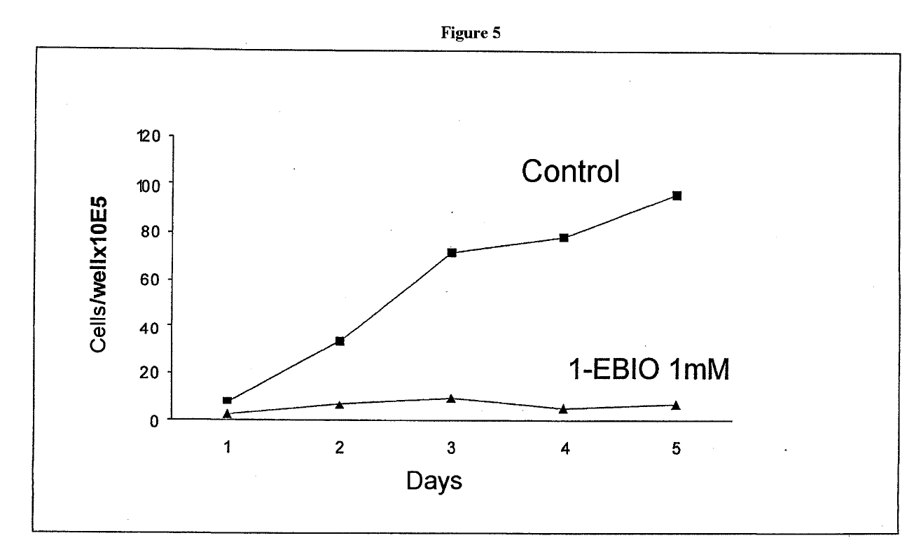

As can be seen from FIG. 5 (n=3), both the addition of one of the two inhibitors and the addition of the hIK1 activator inhibited proliferation while the growth of control cells, to which no modulators had been added, remained unimpaired. The addition of clotrimazole and 1-EBIO at the given concentration led to complete inhibition; by contrast, growth which was only retarded was observed when 200 nM charybdotoxin were added.

The results of the experiment show that modulators of the hIK1 channel affect both parameters of keratinocyte activity, i.e. proliferation and differentiation, simultaneously. Interestingly, this applies in the same way both to activators and to inhibitors: addition leads to inhibition of proliferation and differentiation. This can be explained by the known role of the ion channel in the cell cycle: presumably, phases of both greater opening and of closing are required for passing through a cell cycle. Consequently, both prolonged activation and prolonged inhibition of the activity of the ion channel would lead to inhibition of proliferation, thereby corresponding to the experimental observations. A comparable effect on differentiation can be deduced in an equivalent manner. The discovery of modulators which inhibit both differentiation and proliferation of the keratinocytes provides the possibility, for the first time, of developing completely novel active compounds. Modulating different activities of hIK1 can consequently give rise to special, and completely new, therapeutic possibilities for treating and/or preventing diseases which are connected with disturbed keratinocyte activity. Thus, modulating the activity of hIK1, for example, is particularly advantageous precisely for treating psoriasis, where the cause of the disease is the result of a combination of cell hyperproliferation and the reduced apoptosis of differentiated cells. Particular preference is given to derivatives of the modulators which can be employed at lower concentration, for example DCEBIO (5,6-dichloro-1-ethyl-1,3-dihydro-2H-benzimidazol-2-one), as dichloro derivative of 1-EBIO. An increase in the quantity of the IK channel is correspondingly preferred for treating wound healing disturbances, where the aim is to stimulate keratinocyte proliferation. This can be effected, for example, by gene therapy using a gene-therapy vector which contains a nucleic acid which encodes an IK channel which can be used in accordance with the invention.

It will be evident to the skilled person, that the compositions and processes according to the invention can be modified in many different ways. The intention therefore is that the present invention should also cover those changes and variations which come within the protected scope of the claims and their equivalents.

The priority application DE 100 65 475.4 was filed on Dec. 28, 2000 and the priority application U.S. 60/277,453 was filed on Mar. 20, 2000. All the publications which are cited here are hereby incorporated in their entirety by reference.

TABLE 1

| | | | | SEQ ID | | SEQ |
|---|---|---|---|---|---|---|
| No. | NAME | Organism | PROTEIN | No. | cDNA | ID No. |
| 1. | hIK1 | Homo | trEMBL: | 3 | EMBL: | 1 |
| | | Sapiens | O15554 | | AF000972 | |
| 2. | mIK1 | Mus | trEMBL: | 4 | EMBL: | 2 |
| | | musculus | O89109 | | AF042487 | |

TABLE 2

| | Rel. quantity |
|---|---|
| Wound healing state | of mIK1 cDNA |
| Intact skin | 1.00 |
| Control animals | |
| Wound | 3.00 |
| Control animals | |
| Intact skin | 1.20 |
| Dexamethasone-treated animals | |
| Wound | 2.68 |
| Dexamathasone-treated animals | |
| Intact skin | 0.52 |
| Young animals | |
| Wound | 2.06 |
| Young animals | |
| Intact skin | 1.45 |
| Old animals | |
| Wound | 1.55 |
| Old animals | |
| Intact skin | 1.00 |
| Diabetes control animals (C57B/Ks) | |
| Wound | 2.45 |
| Diabetes control animals (C57B/Ks) | |
| Intact skin | 0.41 |
| Diabetes animals (C57B/Ks-db/db/Ola) | |
| Wound | 2.04 |
| Diabetes animals(C57B/Ks-db/db/Ola) | |

TABLE 3

| Time after | Rel. quantity |
|---|---|
| wounding | of mIK1 cDNA |
| Intact skin | 1.00 |
| 1 h | 1.03 |
| 7 h | 1.71 |
| 15 h | 2.24 |
| 24 h | 1.86 |
| 3 d | 2.71 |
| 5 d | 2.59 |
| 7 d | 2.43 |
| 14 d | 2.24 |

TABLE 4

| | Rel. quantity |
|---|---|
| Biopsy | of hIK1 cDNA |
| Patient pool 1: | 1.00 |
| Intact skin | |
| Healthy patients | |
| Patient pool 2: | 1.32 |
| Day 1 wound | |
| Healthy patients | |
| Patient pool 3: | 0.64 |
| Day 5 wound | |
| Healthy patients | |
| Patient pool 4: | 0.83 |
| Intact skin | |
| Ulcer patients | |
| Patient pool 4: | 0.42 |
| Wound edge | |
| Ulcer patients | |
| Patient pool 4: | 0.81 |
| Wound bottom | |
| Ulcer patients | |

TABLE 5

| | Rel. quantity of |
|---|---|
| Organ | hIK cDNA |
| Skin | 0.54 |
| Kidney | 0.41 |
| Liver | 0.23 |
| Brain | 0.39 |
| Heart | 0.36 |
| Spleen | 0.66 |
| Skeletal muscle | 0.28 |
| Small intestine | 0.56 |
| Lung | 0.58 |
| Fat tissue | 0.58 |

1. A method for diagnosing, preventing, and/or treating a disease which is connected with disturbed keratinocyte activity or for identifying pharmacologically active substances, the method comprising using at least one polypeptide as depicted in one of SEQ ID No. 3 or SEQ ID No. 4, or functional variants thereof, or of a nucleic acid encoding these, or variants thereof, or of at least one antibody which is directed against a polypeptide as depicted in SEQ ID No. 3 or SEQ ID No. 4, or functional variants thereof.

2. The method as claimed in claim 1, characterized in that the disease which is connected with disturbed keratinocyte activity is a wound, in particular psoriasis.

3. The method as claimed in either of claims 1 and 2, characterized in that the nucleic acid is present in the form of its antisense sequence.

4. The method as claimed in either of claims 1 and 2, characterized in that the functional variant is a fusion protein.

5. A method for diagnosing, preventing, and/or treating a disease which is connected with disturbed kerotinocyte activity or for identifying pharmacologically active substances, the method comprising using a host cell which harbors at least one vector and/or at least one knock-out gene construct, which contains a nucleic acid or variants thereof which encode a polypeptide, or functional variants thereof, as depicted in SEQ ID No. 3 or SEQ ID No. 4.

6. The method as claimed in claim 5, characterized in that the host cell is a skin cell.

7. The method as claimed in claim 6, characterized in that the host cell is a keratinocyte.

8. A method for diagnosing, preventing, and/or treating a disease which is connected with disturbed kerotinocyte activity, the method comprising using a modulator of a polypeptide as depicted in SEQ ID No. 3 or SEQ ID No. 4, or functional variants thereof.

9. The method as claimed in claim 8, characterized in that the modulator contains at least one antibody which is directed against a polypeptide as depicted in SEQ ID No. 3 or SEQ ID No. 4, or functional variants thereof.

10. The method as claimed in claim 8 or 9, characterized in that the modulator is an inhibitor.

11. The method as claimed in claim 10, characterized in that the inhibitor is a symmetric or asymmetric derivative of 1,4-dihydropyridine-3,5-dicarboxylic acid in accordance with the following general formula (I) 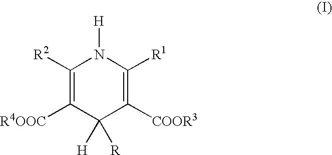
or a pharmaceutically tolerated salt, an oxide or a hydrate thereof;
where
R is an alkyl group or cycloalkyl group; which can be substituted, once or more than once, by substituents selected from the group consisting of halogens,
or R is a monocyclic or polycyclic aryl group, where the aryl group can be substituted, once or more than once, by a substituent selected from a halogen, trifluoromethyl (—CF₃), nitro (—NO₂), cyano (—CN), azido (—N₃), a group of the formula —S(O)ₙ-alkyl, —S(O)ₙ—NH-alkyl or —S(O)ₙ—N-(alkyl)₂, where n can preferably be 0, 1 or 2, an alkyl group, a cycloalkyl group, an alkoxy group, a trifluoromethyloxy group (—OCF₃), a carboxyl group (—COOH), a group of the formula —COO-alkyl, a carbamoyl group (—CONH₂), and a group of the formula—CONH-alkyl or CON(alkyl)₂;
or R is a monoheterocyclic or polyheterocyclic group, where the heterocyclic group can be substituted, once or more than once, by an alkyl group, an alkoxy group, a carboxyl group (—COOH), a group of the formula —COO-alkyl, and/or a group of the formula —COO-phenyl;
and R¹, R², R³ and R⁴ are, independently of each other, hydrogen, an alkyl group, a cycloalkyl group, an alkenyl group, an alkynyl group, an alkoxy group, a phenyl group, a phenylalkyl group, a furanyl group, a furanylalkyl group, a pyridyl group or a pyridylalkyl group.

12. The method as claimed in claim 10, characterized in that the inhibitor is a derivative or metabolite of clotrimazole in accordance with the following general formula (II) 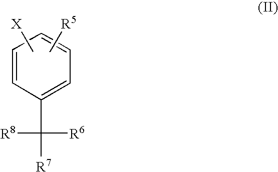
or a pharmaceutically tolerated salt, an oxide or a hydrate thereof;
where
X is a halogen, a trifluoromethyl group, a nitro group or a cyano group;
R⁵ is hydrogen, a halogen, a hydroxyl group, an alkyl group, a cycloalkyl group, an alkoxy group or an alkyloxy group;
R⁶ is hydrogen or a phenyl group, where the phenyl group can be substituted, once or more than once, by substituents selected from a halogen and hydroxyl group;
R⁷ is hydrogen, a halogen, a hydroxyl group, alkyl group or alkoxy group;
R⁸ is a group of the formula —Y—CH₂—R⁹, where Y is oxygen (—O—) or sulfur (—S—); a group of the formula ═NO—CH₂—R⁹; a group of the formula —O-phenyl-CH═CH₂; a group of the formula —CH₂—CH(CH₃)—S-phenyl, where the phenyl group can be substituted, once or more than once, by substituents selected from a halogen and hydroxyl group; or a phenyl group, where the phenyl group can be substituted, once or more than once, by substituents selected from a halogen and hydroxyl group; and where R⁹ is an ethenyl group (CH₂═CH—); a phenyl group, where the phenyl group can be substituted, once or more than once, by substituents selected from a halogen and hydroxyl group; a phenyl-S-phenyl group, a group of the formula CH₂—O-phenyl, where the phenyl group can be substituted, once or more than once, by substituents selected from a halogen and hydroxyl group; or a group of the general formula (VII), 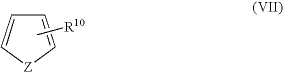
where Z is S, O or N;
and R¹⁰ is hydrogen, a halogen or a hydroxyl group.

13. The method as claimed in claim 8, characterized in that the modulator is an oxime derivative in accordance with the following general formula (III) 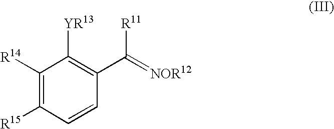
or a pharmaceutically tolerated salt, an oxide or a hydrate thereof;
where
Y is oxygen, sulfur or an amino group, preferably an amino group of the formula NHR¹³;
R¹¹ is hydrogen; an alkyl group; a cycloalkyl group; a hydroxyl group; an alkoxy group; an acyl group; a phenyl group or a benzyl group, where the group, in particular the phenyl group or benzyl group, can be substituted, once or more than once, by substituents selected from a halogen, —CF₃, —NO₂, —CN, alkyl group, cycloalkyl group, hydroxyl group and alkoxy group; a group of the formula CH₂CN; a group of the formula CH₂CO₂R′, where R′ is hydrogen or an alkyl group; a group of the formula CH₂CONR^{IV}R^{V}, where R^{IV} and R^{V} are, independently of each other, hydrogen or an alkyl group; or a group of the formula —CH₂C(═NOH)NH₂;
R¹² is hydrogen; an alkyl group; a cycloalkyl group; a phenyl group or a benzyl group, where the group, in particular the phenyl group or benzyl group, can be substituted, once or more than once, by substituents selected from a halogen, —CF₃, —NO₂, —CN, alkyl group, cycloalkyl group, hydroxyl group and alkoxy group;
R¹³ is hydrogen; an alkyl group; a cycloalkyl group; a phenyl group or a benzyl group, where the group, in particular the phenyl group and benzyl group, can be substituted, once or more than once, by substituents selected from a halogen, —CF₃, —NO₂, —CN, alkyl group, cycloalkyl group, hydroxyl group and alkoxy group; and
R¹⁴ and R¹⁵ are, independently of each other, hydrogen; a halogen; —CF₃; —NO₂; —CN; an alkyl group; an alkoxy group; a phenyl group or a benzyl group, where the group, in particular the phenyl group or benzyl group, can be substituted, once or more than once, by substituents selected from a halogen, —CF₃, —NO₂, —CN, alkyl group, cycloalkyl group, hydroxyl group and alkoxy group; or are a group of the formula —SO₂NR″′, where R″ and R′″ are, independently of each other, hydrogen or an alkyl group;
or R¹⁴ and R¹⁵ together form an additional 4-membered to 7-membered fused ring, where the fused ring can be aromatic or partially saturated and can be substituted, once or more than once, by substituents selected from a halogen, —CF₃, —NO₂, —CN and a group of the formula —SO₂NR″R′″, where R″ and R′″ are, independently of each other, hydrogen or an alkyl group.

14. The method as claimed in claim 8, characterized in that the modulator is a chemical compound in accordance with the following general formula (IV): 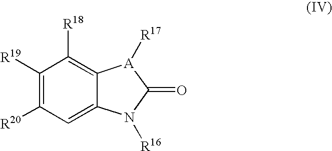
or a pharmaceutically tolerated salt, an oxide or a hydrate thereof;
where
A is oxygen, sulfur or nitrogen;
R¹⁶ is hydrogen; an alkyl group; a cycloalkyl group; a hydroxyl group; an alkoxy group; an acyl group; a phenyl group or a benzyl group, where the group, in particular the phenyl group or benzyl group, can be substituted, once or more than once, by substituents selected from a halogen, —CF₃, —NO₂, —CN, alkyl group, cycloalkyl group, hydroxyl group and alkoxy group; a group of the formula CH₂CN; a group of the formula CH₂CO₂R′, where R′ is hydrogen or an alkyl group; a group of the formula CH₂CONR^{IV}R^{V}, where R^{IV} and R^{V}; are, independently of each other, hydrogen or an alkyl group; or a group of the formula —CH₂C(═NOH)NH₂;
R¹⁷ is hydrogen; an alkyl group; a cycloalkyl group; a phenyl group or a benzyl group, where the group, in particular the phenyl group or benzyl group, can be substituted, once or more than once, by substituents selected from a halogen, —CF₃, —NO₂, —CN, alkyl group, cycloalkyl group, hydroxyl group and alkoxy group;
R¹⁸, R¹⁹ and R²⁰ are, independently of each other, hydrogen; a halogen, —CF₃; —NO₂; —CN; an alkyl group; an alkoxy group; a phenyl group or a benzyl group, where the group, in particular the phenyl group or benzyl group, can be substituted, once or more than once, by substituents selected from a halogen, —CF₃, —NO₂, —CN, alkyl group, cycloalkyl group, hydroxyl group and alkoxy group; or a group of the formula —SO₂NR″R′″, where R″ and R′″ are, independently of each other, hydrogen or an alkyl group;
or R²⁰ is defined as above and R¹⁸ and R¹⁹ together form an additional 4-membered to 7-membered fused ring, where the fused ring can be heterocyclic, can be aromatic, saturated or partially saturated, and optionally can be substituted, once or more than once, by substituents selected from a halogen, —CF₃, —NO₂, —CN and a group of the formula —SO₂NR″R′″, where R″ and R′″ are, independently of each other, hydrogen or an alkyl group.

15. The method as claimed in claim 14, characterized in that the modulator is 1-ethyl-2-benzimidazolinone or 5,6-dichloro-1-ethyl-1,3-dihydro-2H-benzimidazol-2-one.

16. The method as claimed in claim 8, characterized in that the modulator is a chemical compound in accordance with the following general formula (V) 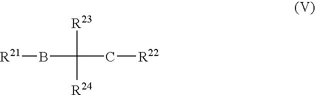
or a pharmaceutically tolerated salt, an oxide or a hydrate thereof;
where
B and C, independently of each other, are a group of the formula —(CH₂)ₙ—, of the formula —(CH₂)ₙ—Y′— (in one of the two directions) or of the formula —(CH₂)ₙ—Y′—(CH₂)ₘ—, where, in this formula, n and m are, independently of each other, 0, 1, 2, 3 or 4, Y′ is O, S or NR′″, where R′″ is hydrogen or an alkyl group;
R²¹ and R²² are, independently of each other, an alkyl, alkenyl, alkynyl, cycloalkyl, amino, trihalomethyl, nitro, cyano, phenyl or a group of the formula OR′, —SR′, —R′OR″, —R′SR″, —C(O)R′, —C(S)R′, —C(O)OR′, —C(S)OR′, —C(O)SR′, —C(S)SR′, —C(O)NR′(OR″), —C(S)NR′(OR″), —C(O)NR′(SR″), —C(S)NR′(SR″), —CH(CN)₂, —C(S)NR′R″, —C(O)NR′R″, —CH[C(O)R′]₂, —CH[C(S)R′]₂, —CH[C(O)OR′]₂, —CH[C(S)OR′]₂, —CH[C(O)SR′]₂, —CH[C(S)SR′]₂, CH₂OR′, CH₂SR′, —NR′C(O)R″, or OC(O)R′;
or is an unsaturated, partially saturated or saturated monocyclic or polycyclic group, an aralkyl group or heteroalkyl group, where the monocyclic or polycyclic groups, aralkyl groups or heteroalkyl groups can be substituted, once or more than once, by a group consisting of halogen, trihalomethyl, alkyl, alkenyl, alkynyl, amino, nitro, cyano or amido, or a group of the formula —R′, —OR′, SR′, —R′OR″, —R′SR″, —C(O)R′, —C(S)R′, —C(O)OR′, —C(S)OR′, —C(O)SR′ or —C(S)SR′, or a phenyl group or phenoxy group, where the group, in particular the phenyl group or phenoxy group, can be substituted, once or more than once, by a group consisting of a halogen, trihalomethyl, alkyl, alkenyl, alkynyl, amino, nitro, cyano or amido, or a group of the formula —R′, —OR′, SR′, —R′OR″, —R′SR″, —C(O)R′, —C(S)R′, —C(O)OR′, —C(S)OR′, —C(O)SR′, —C(S)SR′, —NR′C(O)R″ or OC(O)R′;
where
R′ and R″ are, independently of each other, can be substituted, once or more than once, by a group selected from hydrogen, alkyl, alkenyl, alkynyl, cycloalkyl, alkoxy or phenyl, where appropriate substituted, or a group of the formula NR′″R″″, where R′″ and R″″ independently of each other, are hydrogen or alkyl;
R²³ and R²⁴ are, independently of each other, alkyl, alkenyl, alkynyl, cycloalkyl, amino, trihalomethyl, nitro, cyano, or phenyl, or a group of the formula OR′, —SR′, —R′OR″, —R′SR″, —C(O)R′, —C(S)R′, —C(O)OR′, —C(S)OR′, —C(O)SR′, —C(S)SR′, —C(O)NR′(OR″), —C(S)NR′(OR″), —C(O)NR′(SR″), —C(S)NR′(SR″), —CH(CN)₂, —C(S)NR′R″, —C(O)NR′R″, —CH[C(O)R′]2, —CH[C(S)R′]₂, —CH[C(O)OR′]₂, —CH[C(S)OR′]₂, —CH[C(O)SR′]₂, —CH[C(S)SR′]₂, CH₂OR′, CH₂SR′, —NR′C(O)R″, or OC(O)R′;
where
R′ and R″ are, independently of each other, hydrogen, alkyl, alkenyl, alkynyl, cycloalkyl, alkoxy or phenyl or a group of the formula NR′″R″″, where R′″ and R″″ are, independently of each other, hydrogen or alkyl;
or R²³ and R²⁴ together form an unsaturated, partially saturated or completely saturated monocyclic or polycyclic group or a monoheterocyclic or polyheterocyclic group, where the monocyclic or polycyclic groups can be substituted, once or more than once, by a group consisting of a halogen, trihalomethyl, alkyl, alkenyl, alkynyl, amino, nitro, cyano or amido, or a group of the formula —R′, —OR′, SR′, —R′OR″, —R′SR″, —C(O)R′, —C(S)R′, —C(O)OR′, —C(S)OR′, —C(O)SR′, —C(S)SR′, or a phenyl group or phenoxy group, where the group, in particular the phenyl group or phenoxy group, can be substituted, once or more than once, by a group consisting of a halogen, trihalomethyl, alkyl, alkenyl, alkynyl, amino, nitro, cyano or amido, or a group of the formula —R′, —OR′, SR″, —R′OR″, —R′SR″, —C(O)R′, —C(S)R′, —C(O)OR′, —C(S)OR′, —C(O)SR′, —C(S)SR′, —NR′C(O)R″ or OC(O)R′;
where
R′ and R″ are, independently of each other, hydrogen, alkyl, alkenyl, alkynyl, cycloalkyl, alkoxy, phenyl or a group of the formula NR″′R″″, where R′″ and R″″ are, independently of each other, hydrogen or alkyl.

17. The method as claimed in claim 8 or 9, characterized in that the modulator is an activator.

18. The method as claimed in claim 17, characterized in that the activator is an isatin derivative in accordance with the following general formula (VI) 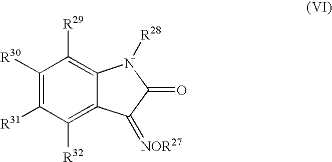
or a pharmaceutically tolerated salt, an oxide or a hydrate thereof;
where
R²⁷ is hydrogen; an alkyl group; a cycloalkyl group; an acyl group; a phenyl group or benzyl group, where the group, in particular the phenyl group or benzyl group, can be substituted, once or more than once, by a group selected from a halogen, —NO₂, —CN, —CF₃, alkyl group, cycloalkyl group, hydroxyl group and alkoxy group; a group of the formula —CH₂CN; a group of the formula —CH₂CO₂R′, where R′ is hydrogen or an alkyl group; a group of the formula —CH₂CONR^{IV}R^{V}, where R^{IV} and R^{V} are, independently of each other, hydrogen, alkyl, a phenyl group or benzyl group, where the phenyl group or benzyl group can be substituted, once or more than once, by a halogen and/or alkyl; or R^{IV} and R^{V}, together with the N atom to which they are bonded, form a 4-membered to 7-membered monocyclic ring, where the heterocyclic group can be substituted, once or more than once, by a group selected from a halogen, alkyl, cycloalkyl, alkyloxy, cycloalkyloxy, phenyl or benzyl; or a group of the formula —CH₂C(═NOH)NH₂;
R²⁸ is hydrogen; an alkyl group; a cycloalkyl group; a group of the formula —CH₂CO₂R′, where R′ is hydrogen or an alkyl group; or is a phenyl group or benzyl group, where the phenyl group or benzyl group can be optionally substituted, once or more than once, by substituents selected from a halogen, —NO₂, —CN, CF₃, alkyl, cycloalkyl, hydroxyl and alkoxy; and
R²⁹, R³⁰, R³¹ and R³² are, independently of each other, hydrogen; a halogen; —NO₂; —CN; CF₃; alkyl; an alkoxy group; a phenyl group or benzyl group, where the phenyl group or benzyl group can be substituted, once or more than once, by substituents selected from a halogen, —NO₂, —CN, CF₃, alkyl, cycloalkyl, hydroxyl and alkoxy; or a group of the formula —SO₂NR″R′″, where R″ and R′″ are, independently of each other, hydrogen or an alkyl group;
or R³¹ and R³² are defined as above, and R²⁹ and R³⁰ together form an additional 4-membered to 7-membered fused ring, where the fused ring can be aromatic, partially saturated or saturated and the fused ring can be substituted, once or more than once, by substituents selected from a halogen, —NO₂, —CN, CF₃ and a group of the formula —SO₂NR″R′″, where R″ and R′″ are, independently of each other, hydrogen or an alkyl group.

19. The method as claimed in claim 8, characterized in that the disease which is connected with disturbed keratinocyte activity is a wound, in particular psoriasis.

20. The method as claimed in claims 1, 5, or 8, characterized in that, for diagnosing diseases which are connected with disturbed keratinocyte activity, at least one polypeptide as depicted in SEQ ID No. 3 or as depicted in SEQ ID No. 4, or a functional variant thereof, or at least one nucleic acid encoding it, or at least one antibody which is directed against a polypeptide as depicted in SEQ ID No. 3 or SEQ ID No. 4, or functional variants thereof, or at least one modulator of a polypeptide as depicted in SEQ ID No. 3 or SEQ ID No. 4 or functional variants thereof, is combined with suitable additives and auxiliary substances.

21. The method as claimed in claim 20, characterized in that the nucleic acid is employed in the form of a probe, preferably a DNA probe.

22. The method as claimed in 1, 5, or 8, characterized in that, for preventing and/or treating diseases which are connected with disturbed keratinocyte activity, at least one polypeptide as depicted in SEQ ID No. 3 or SEQ ID No. 4, or a functional variant thereof, or at least one nucleic acid encoding it, or at least one antibody which is directed against a polypeptide as depicted in SEQ ID No. 3 or SEQ ID No. 4, or functional variants thereof, or at least one modulator of a polypeptide as depicted in SEQ ID No. 3 or SEQ ID No. 4 or functional variants thereof is combined with suitable additives and auxiliary substances.

23. The method as claimed in claim 22, characterized in that the nucleic acid is an antisense oligonucleotide, a small interfering RNA molecule (siRNA) or a ribozyme.

24. A method for performing an analysis in connection with a disease which are is connected with disturbed keratinocyte activity, the method comprising using an array, which is fixed on a solid support, and characterized in that the array contains at least one polypeptide as depicted in SEQ ID No. 3 or SEQ ID No. 4, or a functional variant thereof, or at least one nucleic acid encoding it, or at least one antibody which is directed against a polypeptide as depicted in SEQ ID No. 3 or SEQ ID No. 4, or functional variants thereof.

25. A method for diagnosing psoriasis, the method comprising using a diagnostic agent comprising chlorzoxazone.

26. A method for preventing and/or treating psoriasis, the method comprising administering a pharmaceutical comprising chlorzoxazone.
